(19) United States
(12) Patent Application Publication
(10) Pub. No.: US 2002/0107431 A1
(43) Pub. Date: Aug. 8, 2002

(54) Surrogate sexual partner

(76) Inventor: Rex More, Oak Park, CA (US)

Correspondence Address: Daniel P. McCarthy, Parsons Behle & Latimer, Suite 1800, 201 South Main Street, P.O Box 45898, Salt Lake City, UT, 84145-0898, US

(21) Appl. No.: 09/733,838

(22) Filed: Dec. 8, 2000

(51) Int. Cl. A61F 5/00

(52) U.S. Cl. 600/38

(57) ABSTRACT

A surrogate sexual partner device, structures and materials thereof, uses therefor and methods for making the same are disclosed. Some embodiments of the surrogate sexual partner invention include artificial flesh made from a gelatinous elastomer that closely approximates the consistency and feel of genuine human flesh. Operable orifices may be provided on the surrogate sexual partner device. Some embodiments of the surrogate sexual partner device include an articulating skeleton, spinal, rib, finger, and other bone structures to approximate the feel and maneuverability of a genuine human body. A reciprocating pelvis may be provided for life-like interaction. Hair, skin and eye color, race, body size and proportion may be chosen according to user preference. The surrogate sexual partner device may be constructed to fulfill an individual's fantasy for a dream partner, to resemble a celebrity, or to have any other desired physical appearance.

### I. BACKGROUND OF INVENTION

A. Field of Invention

The invention relates to the field of sexual therapy, marriage counseling, psychiatric and psychological counseling, sexual devices and sexual aids.

B. Description of Related Art

Societal conditions today present many problems to human sexuality. The risk of contracting sexually-transmitted diseases, or social inhibition deters many persons from engaging in normal sexual activity, resulting in pent-up desires and possibly aggressive or aberrant behavior. As our society focuses heavily on beauty and fitness, many males feel that the sexual partners they are able to find do not fulfill their ideals, resulting in sexual dissatisfaction. Further, incarcerated persons face either masturbation or homosexual activity as the only way to satisfy their sexual urges.

There is also a need for a life-like and realistic surrogate sexual partner for use in marriage counseling, sexual therapy, psychiatric counseling and psychological counseling. Without a very life-like sexual surrogate partner, the counseling is less likely to be effective.

There is also a need for a realistic and life-like surrogate sexual partner for adult amusement.

In the background art, there were many crude sexual aids, including inflatable sex dolls, plastic vaginas, medical and educational demonstration models, mannequins, CPR (cardio-pulmonary resusitation) artificial insemination devices, and sperm collection mannequins. Those devices were inadequate, unrealistic, unnatural to the touch, and ineffective.

With the development of product called REALDOLL® by Abyss Creations of San Marcos, Calif. sexual surrogate partners achieved a much higher degree of realism than ever before with the use of silicone flesh supported by an interior articulating skeleton, and life-cast hands and feet (i.e., molded directly from human hands and feet). Although REALDOLL®1 is of very high quality, the crippling drawback is also its main strength; silicone, Silicone formulations soft enough to feel lifelike are necessarily weaker. The poor tear propagation properties of silicone are well known to those versed in the art. In one instance, a REALDOLL®1 owner who wanted to move his sexual surrogate partner across the carpet took hold of her feet (she was on her stomach), and in the distance of a few feet had torn her breasts off.

Another disadvantage of silicone is its high cost. The silicone required to create one sexual surrogate partner costs about $2000, awhile the triblock copolymer gel of the current invention costs less than $100 at the time of writing of this document (a 95% cost savings on the main ingredient alone).

### II. SUMMARY AND OBJECTS OF INVENTION

It is an object of the invention to provide a surrogate sexual partner device, structures and materials thereof, and methods for making the same, as disclosed herein and as recited in the claims. Therapeutic objects of the invention that can be achieved, as desired, include performing sexual therapy, marriage counseling, psychiatric counseling and psychological counseling using a lifelike but artificial sexual partner. Other objects that can be achieved using the invention include providing for sexual activity without a human partner, sexual activity without the risk of disease transmission, sexual activity with a fantasy partner such as a celebrity likeness, safe sexual activity for incarcerated persons, sexual activity with a spouse-likeness during times of spousal absence or after a spouse has aged, sexual activity for socially-inhibited persons, and adult amusement. The invention also addresses the need for life-like models for CPR and medical examination instruction.

Another object of the invention is to achieve the above-mentioned objects at reduced material cost compared to some of the prior art.

Additional objects, features and advantages of the invention will become apparent to the reader upon reviewing the specification and the appended drawings.

### III. BRIEF DESCRIPTION OF THE DRAWINGS

FIG. 1 depicts a full body frontal view of an exemplary surrogate sexual partner of the invention.

FIG. 2 depicts a full body side view of an exemplary surrogate sexual partner of the invention.

FIG. 3 depicts a head assembly side view of a head of an exemplary surrogate sexual partner of the invention.

FIG. 4 depicts a head assembly front view of a head of an exemplary surrogate sexual partner of the invention.

FIG. 5 depicts a pelvis assembly front view of an exemplary surrogate sexual partner of the invention.

FIG. 6 depicts a pelvis assembly side view of an exemplary surrogate sexual partner of the invention.

FIG. 7 depicts a spine assembly front view of an exemplary surrogate sexual partner of the invention.

FIG. 8 depicts a spine assembly side view of an exemplary surrogate sexual partner of the invention.

FIG. 9 depicts a rib assembly front view of an exemplary surrogate sexual partner of the invention.

FIG. 10 depicts a rib assembly side view of an exemplary surrogate sexual partner of the invention.

FIG. 11 depicts a rib limiter side view of an exemplary surrogate sexual partner of the invention.

FIG. 12 depicts a hand assembly side view of an exemplary surrogate sexual partner of the invention.

FIG. 13 depicts a spine assembly top view of an exemplary surrogate sexual partner of the invention.

FIG. 14 depicts a joint assembly of an exemplary surrogate sexual partner of the invention.

### IV. DETAILED DESCRIPTION OF A PREFERRED EMBODIMENT

A. Materials for Making Fleshy Portions of the Invention

1. Elastomer Materials

The compositions of the preferred gelatinous elastomers are generally soft compounds which include an elastomeric polymer component and a plasticizer component.

Tri-block polymers of the general configuration A-B-A, wherein the A represents a crystalline polymer such as a monoalkenylarene polymer and the B is an elastomeric polymer, are used in the gelatinous elastomers. A plasticizer such as an oil is used to extend the A-B-A tri-block copolymers.

The elastomer component of some preferred materials used in the invention includes a triblock polymer of the general configuration A-B-A, wherein the A represents a crystalline polymer such as a monoalkenylarene polymer, including but not limited to polystyrene and functionalized polystyrene, and the B is an elastomeric polymer such as poly(ethylene/butylene), poly(styrene-ethylene-butylene-styrene), hydrogenated poly(isoprene), hydrogenated poly(butadiene), hydrogenated poly(isoprene+butadiene), poly(ethylene/propylene) or hydrogenated poly(ethylene/butylene+ethylene/propylene), or others. The A components of the material link to each other to provide strength, while the B components provide elasticity. Polymers of greater molecular weight are achieved by combining many of the A components in the A portions of each A-B-A structure and combining many of the B components in the B portion of the A-B-A structure, along with the networking of the A-B-A molecules into large polymer networks. A preferred elastomer for making gel material for use in the invention is high weight average molecular weight elastomer and oil compound.

The elastomeric B portion of some preferred A-B-A polymers has an exceptional affinity for most plasticizing agents, including but not limited to several types of oils. When the network of A-B-A molecules is denatured, plasticizers which have an affinity for the B block can readily associate with the B blocks. Upon renaturation of the network of A-B-A molecules, the plasticizer remains highly associated with the B portions, resulting in a very low durometer, very strong elastomer having little or no oil bleed even at very high oil:elastomer ratios.

Some elastomers used in the invention include KRATON G1651 sold by Shell and SEPTON 4045, SEPTON 4077, SEPTON 2055, SEPTON 2006, SEPTON 8006 and SEPTON 4033 from Kuraray. Other materials with chemical and physical characteristics similar to those of SEPTON 4055 include other A-B-A triblock copolymers which have a hydrogenated midblock polymer that is made up of at least about 30% isoprene monomers and at least about 30% butadiene monomers, the percentages being based on the total number of monomers that make up the midblock polymer. Similarly, other A-B-A triblock copolymers which have a hydrogenated midblock polymer that is made up of at least about 30% ethylene/propylene monomers and at least about 30% ethylene/butylene monomers, the percentages being based on the total number of monomers that make up the midblock polymer, are materials with chemical and physical characteristics similar to those of SEPTON 4055.

Triblock copolymer gels especially suitable for use in forming the gel components of the composites of the invention include: SEBS gels; examples include: (a) Kraton G 1651, G 1654X gels; (b) Kraton G 4600 gels; (c) Kraton G 4609 gels; other less suitable SEBS oil gels: examples include: (d) Tuftec H 1051 gels; (e) Tuftec H 1041 gels; (f) Tuftec H 1052 gels. Gels made from blends (polyblends) of (a)-(f) with other polymers and copolymers include: SEBS-SBS gels; SEBS-SIS gels; SEBS-(SEP) gels; SEBS-(SB)n gels; SEBS-(SEB)n gels; SEBS-(SEP)n gels; SEBS-(SI)n gels; SEBS-(SI) multiarm gels; SEBS-branched copolymers gels; SEBS-star shaped copolymer gels; gels made from blends of (a)-(f) with other homopolymers include: SEBS/polystrene gels; SEBS/polybutylene gels; SEBS/polyethylene gels; SEBS/polypropoylene gels. Other suitable thermoplastic elastomers in blends suitable for making gels include SEP/SEBS oil gels, SEP/SEPS oil gels, SEP/SEPS/SEB oil gels, SEPS/SEBS/SEP oil gels, SEB/SEBS, EB-EP/SEBS, SEBS/EB, SEBS/EP, SEPS/SEB, etc.

A more detailed list of commercial elastomers useful in making gels includes Shell Kratons D1101, D1102, D1107, D1111, D1112, D1113X, D1114X, D1116, D1117, D1118X, D1122X, D1125X, D1133X, D1135X, D1184, D1188X, D1300X, D1320X, D4122, D4141, D4158, D4240, G1650, G1651.G1652, G1657, G1701X, G1702X, G1726X, G1750X, G1765X, FG1901X, FG1921X, D2103, D2109, D2122X, D3202, D3204, D3226, D5298, D5999X, D7340, G1654X, G2701, G2703, G2705, G1706, G2721X, G7155, G7430, G7450, G7523X, G7528X, G7680, G7705, G7702X, G7720, G7722X, G7820, G7821X, G7827, G7890X, G7940; Kuraray's SEP/SEPS/SEEPS: Nos. 1001(SEP), 2002(SEPS), 2003(SEPS), 2023(SEPS), 2043(SEPS), 2063(SEPS), 2005(SEPS), 2006(SEPS), 1050(SEP), 2103(SEPS), 2104(SEPS), 2105(SEPS), and 4055(SEEPS) manufactured by Kuraray Co., Ltd., wherein SEP is made from hydrogentaed styrene isoprene di-block copolymer(SI), SEPS is made from hydrogentaed styrene isoprene triblock copolymer(SIS), and SEEPS is made from hydrogenated styrene isoprene/butadiene block copolymer or more specifically made from hydrogenated styrene block polymer with 2-methyl-1,3-butadiene and 1,3-butadiene.

The most preferred gels forming the composites of the invention comprise a high viscosity triblock copolymers which have the more general configuration A-B-A wherein each A is a crystalline polymer end block segment of polystyrene; and B is a elastomeric polymer center block segment of poly(ethylene-butylene). The poly(ethylene-butylene) and polystyrene portions are incompatible and form a two-phase system consisting of sub-micron domains of glassy polystyrene interconnected by flexible poly(ethylene-butylene) chains. These domains serve to crosslink and reinforce the structure. This physical elastomeric network structure is reversible, and heating the polymer above the softening point of polystyrene temporarily disrupt the structure, which can be restored by lowering the temperature.

Some suitable gels for making the surrogate sexual partner of the invention include: (I) 100 parts by weight of one or more high viscosity triblock copolymers of the general configurations poly(styrene-ethylene-butylene-styrene), poly(styrene-ethylene-butylene)[n] or a mixture thereof, wherein the subscript n denotes an integer; (II) a selected amount of a plasticizer sufficient to achieve a gel rigidity of from less than about 2 gram Bloom to about 1,800 gram Bloom. Optionally, the copolymer and plasticizer of the gel may be combined with various additives as described below including one or more of a selected polymer or copolymer selected from the group consisting of poly(styrene-butadiene-styrene), poly(styrene-butadiene), poly(styrene-isoprene-styrene), poly(styrene-isoprene), poly(styrene-ethylene-propylene), poly(styrene-ethylene-propylene-styrene), poly(styrene-ethylene-butylene-styrene), poly(styrene-ethylene-butylene), poly(styrene-ethylene-propylene)n, poly(styrene-ethylene-butylene)n, maleated poly(styrene-ethylene-propylene-styrene), maleated poly(styrene-ethylene-butylene-styrene), maleated poly(styrene-ethylene-butylene), maleated poly(styrene-ethylene-propylene)n, maleated poly(styrene-ethylene-butylene)n, polystyrene, polybutylene, poly(ethylene-propylene), poly(ethylene-butylene), polypropylene, polyethylene, polyethyleneoxide, poly(dimethylphenylene oxide), copolymers of trifluoromethyl-4,5-difuoro-1,3-dioxole and tetrafluoroethylene, tetrafluoroethylene, polycarbonate, ethylene vinyl alcohol copolymer, polyamide or polydimethylsiloxane; wherein said selected copolymer is a linear, branched, radial, or multiarm copolymer.

Mixtures of elastomers are also useful as the elastomer component of some of the formulations of the present invention. In elastomer mixtures, each elastomer contributes different properties to the material. For example, high strength elastomers are desired to improve the tensile strength and durability of a material. However, some high strength elastomers are very difficult to process with some plasticizers. Thus, in such a case, elastomers which improve the processability of the materials are desirable.

Another preferred elastomeric gel composition comprises a styrene-ethylene propylene-styrene block copolymer, that is highly extended with oil such as mineral oil, such that the ratio of SEBS to oil is carefully controlled to simulate the tactile feel of flesh. Some preferred are SEPS polymers having a number average molecular weight of at least 150,000 to 200.000. The SEPS is highly extended with oil such as mineral oil, so the ratio of SEPS to oil is carefully controlled to simulate the tactile feel of flesh. White mineral oil, naphthenic oils, and synthetic oils such as Shellflex.RTM. 371, manufactured by Shell Chemical Co. are preferred. Other oils include petroleum paraffinic oils, petroleum naphthenic oils, synthetic polybutene oils, synthetic polypropene oils, synthetic polyterpene oils and mixtures thereof.

In particular, the process of extending SEPTON 4055 with paraffinic white mineral oil is improved via a lower melt viscosity by using a small amount of more flowable elastomer such as SEPTON 8006, SEPTON 2005, SEPTON 2006, or SEPTON 4033, to name only a few, without significantly changing the physical characteristics of the material.

In another example of the usefulness of elastomer mixtures in the gel, many elastomers are not good compatibilizers. Thus, the use of small amount of elastomers which improve the uniformity with which a material mixes are desired. KRATON G1701, manufactured by Shell Chemical Company of Houston, Tex., is one such elastomer that is useful when making gels based on SEPTON 4055.

The high viscosity triblock copolymer of the invention can have a broad range of styrene end block to ethylene and butylene center block ratio of approximately about 20:80 or less to about 40:60 or higher. Examples of high viscosity triblock copolymers that can be utilized to achieve one or more of the novel properties of the present invention are styrene-ethylene-butylene-styrene block copolymers (SEBS) available from Shell Chemical Company and Pecten Chemical Company (divisions of Shell Oil Company) under trade designations Kraton-G 1651 Kraton G 1654X, Kraton G 4600, Kraton G 4609 and the like. Other grades of (SEBS) polymers can also be utilized in the present invention provided such SEBS polymers exhibits the required high viscosity. Such SEBS polymers include (high viscosity) Kraton G 1855X which has a Specific Gravity of 0.92, Brookfield Viscosity of a 25 weight percent solids solution in toluene at 25. degree. C. of about 40,000 cps or about 8,000 to about 20,000 cps at a 20 weight percent solids solution in toluene at 25. degree. C.

The styrene to ethylene and butylene weight ratios for these Shell designated polymers can have a low range of 20:80 or less. Although the typical ratio values for Kraton G 1651, 4600, and 4609 are approximately about 33:67 and for Kraton G 1855X approximately about 27:73, Kraton G 1654X (a lower molecular weight version of Kraton G 1651 with somewhat lower physical properties such as lower solution and melt viscosity) is approximately about 31:69, these ratios can vary broadly from the typical product specification values. Shell Technical Bulletin SC: 1393-92 gives solution viscosity as measured with a Brookfield model RVT viscometer at 25. degrec. C. for Kragon G 1654X at 10% weight in toluene of approximately 400 cps and at 15% weight in toluene of approximately 5,600 cps.

An ideal value for the ratio of Kraton to plasicizing oil as defined above for firm tissue is 1:8. while 1:6 to 1:11 are quite acceptable and form gels where the composition has the tactile feel of human flesh, depending on the specific part of the body being replicated. It has also been found that in most cases, depending of course on specific molecular weight SEPS polymers and specific oils, a value of the ratio of 1:4 produces a gelatinous composition that is too hard to simulate the tactile feel of flesh. Also, a value of the ratio of 1:13 is too soft to accomplish the goal of simulating the tactile feel of flesh. Thus the preferred range for the ratio is between about 1:5 and 1:12, and the most preferred range for the ratio, as defined above, is between about 1:6 and 1:11.

The ideal value for the ratio of Kraton to plasicizing oil as defined above for soft tissue is 1:14, while 1:12 to 1:15 are quite acceptable and form gels where the composition has the tactile feel of human flesh, depending on the specific part of the body being replicated. It has also been found that in most cases, depending of course on specific molecular weight SEPS polymers and specific oils, a value of the ratio of 1:10 produces a gelatinous composition that is too hard to simulate the tactile feel of flesh. Also, a value of the ratio of 1:17 is too soft to accomplish the goal of simulating the tactile feel of flesh. Thus the preferred range for the ratio is between about 1:11 and 1:16, and the most preferred range for the ratio, as defined above, is between about 1:12 and 1:15.

Many other elastomers, including but not limited to triblock copolymers and diblock copolymers are also useful in the present invention. Applicant believes that elastomers having a significantly higher molecular weight than the ultra-high molecular weight elastomers useful in the present invention increase the softness of a plasticizer extended material, but decrease its strength. Thus, high to ultra high molecular weight elastomers, as defined above, are desired for use in the material of the present invention due to their strength when combined with a plasticizer.

2. Plasticizers

Plasticizers allow the midblocks of a network of triblock copolymer molecules to move past one another. Thus, Applicant believes that plasticizers, when trapped within the three dimensional web of triblock copolymer molecules, facilitate the disentanglement and elongation of the elastomeric midblocks as a load is placed on the network. Similarly, Applicant believes that plasticizers facilitate recontraction of the elastomeric midblocks following release of the load.

Preferably, the plasticizer component of the material of gel is solely or includes a significant amount of a commercially available oil or mixture of oils. The plasticizer component may include other plasticizing agents, such as liquid oligomers and others, as well. Both naturally derived and synthetic oils are useful in the material of the present invention. Preferably, when used in the gel, a plasticizer increases the percent elongation at break of the elastomer component by at least about a factor of two. For example, when an elastomer having a percent elongation at break of about 1,000 is compounded with a preferred plasticizer, the compound material preferably has a percent elongation at break of at least about 2,000.

Preferably, when used in the gel, a plasticizer decreases the Gram Bloom rigidity of the elastomer component by at least about a factor of two. For example, when an elastomer having a Gram Bloom rigidity of about 1,200 is compounded with a preferred plasticizer, the compound material preferably has a Gram Bloom rigidity of about 600 or less.

A preferred plasticizer component of the gel includes paraffinic white mineral oils, such as those having the brand name DUOPRIME, by Lyondell Lubricants of Houston, Tex., and the oils sold under the brand name TUFFLO by Witco Corporation of Petrolia, Pa. A preferred embodiment of the plasticizer component of the gel includes paraffinic white mineral oil such as that sold under the trade name LP-150 by Witco. Other oils are also useful as plasticizers in compounding the gel. Examples of representative commercially available oils include processing oils such as paraffinic and naphthenic petroleum oils, highly refined aromatic-free or low aromaticity paraffinic and naphthenic food and technical grade white petroleum mineral oils, and synthetic liquid oligomers of polybutene, polypropene, polyterpene, etc., and others. The synthetic series process oils are oligomers which are permanently fluid liquid non-olefins, isoparaffins or paraffins.

Plasticizers particularly preferred for use in practicing the present invention are well known in the art, they include rubber processing oils such as paraffinic and naphthenic petroleum oils, highly refined aromatic-free paraffinic and naphthenic food and technical grade white petroleum mineral oils, and synthetic liquid oligomers of polybutene, polypropene, polyterpene, etc. The synthetic series process oils are high viscosity oligomers which are permanently fluid liquid nonolefins, isoparaffins or paraffins of moderate to high molecular weight.

Examples of representative commercially oils include Amoco Registered TM polybutenes, hydrogenated polybutenes and polybutenes with epoxide functionality at one end of the polybutene polymer: Example of such polybutenes include: L-14 (320 Mn), L-50 (420 Mn), L-100 (460 Mn), H-15 (560 Mn), H-25 (610 Mn), H-35 (660 Mn), H-50 (750 Mn), H-100 (920 Mn), H-300 (1290 Mn), L-14E (27-37 cst £ 100o F. Viscosity), H-300E (635-690 cst £ 210o F. Viscosity), Actipol E6 (365 Mn), E16 (973 Mn), E23 (1433 Mn) and the like. Example of various commercially oils include: ARCO Prime (55, 70, 90, 200, 350, 400 and the like), Duraprime and Tufflo oils (6006, 6016, 6016M, 6026, 6036, 6056, 6206, etc), other white mineral oils include: Bayol, Bernol, American, Blandol, Drakeol, Ervol, Gloria, Kaydol, Litetek, Lyondell (Duraprime 55, 70, 90, 200, 350, 400, etc), Marcol, Parol, Peneteck, Primol, Protol, Sontex, and the like. Generally, plasticizing oils with average molecular weights less than about 200 and greater than about 700 may also be used (e.g. H-300 (1290 Mn)).

The addition of other plasticizers to the materials of the gel is useful for tailoring the physical characteristics of the material of the present invention. For example, characteristics such as oil bleed, tack, tensile strength, and the rate at which a material recovers from deformation may be modified with plasticizers other than oil. When resin is included with oil in a plasticizer mixture to form the gel, the gel exhibits significantly reduced oil bleed. For example, a material which includes about one part elastomer component (such as SEPTON 4055), three parts plasticizing oil (such as LP-150), and about five parts plasticizing resin (such as REGALREZ Registered TM 1018) exhibits infinitesimal oil bleed at room temperature, if any, even when placed against materials with high capillary action, such as paper. Prior art gelatinous elastomers bleed noticeably under these circumstances.

3. Additives

a. Detackifiers

The elastomeric materials used in the present invention may include a detackifier. Tack is not a desirable feature on the exterior surface of a surrogate sexual partner. Soaps, detergents and other surfactants have detackifying abilities and are useful in the present invention. “Surfactants,” as defined herein, refers to soluble surface active agents which contain groups that have opposite polarity and solubilizing tendencies. Surfactants form a monolayer at interfaces between hydrophobic and hydrophilic phases; when not located at a phase interface, surfactants form micelles. Surfactants have detergency, foaming, wetting, emulsifying and dispersing properties. For example, coco diethanolamide, a common ingredient in shampoos, is useful in the present invention as a detackifying agent. Coco diethanolamide resists evaporation, is stable, relatively non-toxic, non-flammable and does not support microbial growth. Many different soap or detergent compositions could be used as well.

Other known detackifiers include glycerin, epoxidized soybean oil, dimethicone, tributyl phosphate, block copolymer polyether, diethylene glycol mono oleate, polyisobutylene, butyl rubber and others, and silicone to name only a few.

b. Antioxidants

A preferred compound used in the present invention may also include additives such as an antioxidant. Antioxidants such as those sold under the trade names IRGANOX 1010 and IRGAFOS 168 by Ciba-Geigy Corp. of Tarrytown, N.Y. are useful by themselves or in combination with other antioxidants in the preferred materials of the present invention.

Antioxidants protect the preferred materials of the gel against thermal degradation during processing which requires or generates heat. In addition, antioxidants provide long term protection from free radicals. A preferred antioxidant inhibits thermo-oxidative degradation of the compound or material to which it is added, providing long-term resistance to polymer degradation.

Heat, light (in the form of high energy radiation), mechanical stress, catalyst residues, and reaction of a material with impurities all cause oxidation of the material. In the process of oxidation, highly reactive molecules known as free radicals are formed and react in the presence of oxygen to form peroxy free radicals, which further react with organic material (hydrocarbon molecules) to form hydroperoxides.

The two major classes of antioxidants are the primary antioxidants and the secondary antioxidants. Peroxy free radicals are more likely to react with primary antioxidants than with most other hydrocarbons. In the absence of a primary antioxidant, a peroxy free radical would break a hydrocarbon chain. Thus, primary antioxidants deactivate a peroxy free radical before it has a chance to attack and oxidize an organic material.

Most primary antioxidants are known as sterically hindered phenols. One example of sterically hindered phenol is the C73H108012 marketed by Ciba-Geigy as IRGANOX 1010, which has the chemical name 3,5-bis(1,1 -dimethylethyl)-4-hydroxybenzenepropanoic acid,2,2-bis[[3-[3,5-bis(dimethylethyl)-4-hydroxyphenyl]-1-oxopropoxy]methyl]1,3-propanediyl ester. The FDA refers to IRGANOX 1010 as tetrakis[methylene(3,5-di-tert-butyl-4-hydroxyhydrocinnimate)]methane. Other hindered phenols are also useful as primary antioxidants in the present invention.

Similarly, secondary antioxidants react more rapidly with hydroperoxides than most other hydro-carbon molecules. Secondary antioxidants have been referred to as hydroperoxide decomposers. Thus, secondary antioxidants protect organic materials from oxidative degradation by hydroperoxides.

Commonly used secondary antioxidants include the chemical classes of phosphites/phosphonites and thioesters, many of which are useful in the materials of the present invention. A hydroperoxide decomposer that can be used is a C42H6303P phosphite known as Tris(2,4-di-tert-butylphenyl)phosphite and marketed by Ciba-Geigy as IRGAFOS 168.

It is known in the art that primary and secondary antioxidants form synergistic combinations to ward off attacks from both peroxy free radicals and hydroperoxides.

Other antioxidants, including but not limited to multi-functional antioxidants, are also useful in the present invention. Multifunctional antioxidants have the reactivity of both a primary and a secondary antioxidant. IRGANOX 1520 D, manufactured by Ciba-Geigy Corp. of Tarrytown, N.Y. is one example of a multifunctional antioxidant.

Preferably, the materials used in the present invention include up to about three weight percent antioxidant, based on the weight of the elastomer component, when only one type of antioxidant is used. When a combination of antioxidants is used, each may comprise up to about three weight percent, based on the weight of the elastomer component. In the presently most preferred embodiment of the present invention, the materials include 2.5 weight percent primary antioxidant and 2.5 weight percent secondary antioxidant, both percentages based on the weight of the elastomer component of the material. Additional antioxidants may be added for severe processing conditions involving excessive heat or long duration at a high temperature.

Use of excess antioxidants reduces or eliminates tack on the exterior surface of the gel. Excess antioxidants tend to migrate to the exterior surface of the material following compounding of the material. Such migration occurs over substantial periods of time, from hours to days or even longer. The migration of antioxidants to the exterior surface of the material is facilitated by lubricants that are not compatible with the A-B-A triblock copolymers useful in the material of the invention, such as linseed oil, castor oil, or others.

3. Flame retardants

Flame retardants may also be added to the gel materials of the present invention. Flame retardants useful in the invention include but are not limited to diatomaceous earth flame retardants, halogenated flame retardants, non-halogenated flame retardants, and volatile, non-oxygen gas forming chemicals.

4. Colorants

Colorants may also be used in the materials of the present invention. Any colorant which is compatible with elastomeric materials may be used in the materials of this invention. Colorants are desired in order to produce a correct skin tone for the race and pigmentation of the surrogate sexual partner.

5. Other additives

Other additives may also be added to the gel material. Additives such as foaming facilitators, tack modifiers, plasticizer bleed modifiers, flame retardants, melt viscosity modifiers, melt temperature modifiers, tensile strength modifiers, and shrinkage inhibitors are useful in specific embodiments of the material of the invention.

Melt temperature modifiers useful in the gel include diblock copolymers of the general configuration A-B and triblock copolymers of the general configuration A-B-A wherein the end block A polymers include functionalized styrene monomers, cross-linking agents, hydrocarbon resins and others.

Other polymers and copolymers (in major or minor amounts) can be melt blended with the copolymers mentioned above without substantially decreasing the desired properties. Such polymers may also be utilized in one or more combinations of the composits of the invention; these include (SBS) styrene-butadiene-styrene block copolymers, (SIS) styrene-isoprene-styrene block copolymers, (low styrene content SEBS) styrene-ethylene-butylene-styrene block copolymers, (SEP) styrene-ethylene-propylene block copolymers, (SEPS) styrene-ethylene-propylene-styrene block copolymers, (SB)n styrene-butadiend and (SEB)n, (SEBS)n, (SEP)n, (SI)n styrene-isoprene multi-arm, branched, and star shaped copolymers and the like. Still, other homopolymers can be utilized in minor amounts; these include: polystyrene, polybutylene, polyethylene, polypropoylene and the like.

Other additives that can be considered for inclusion in the gel include: tetrakis[methylene 3,-(3′5′-di-tertbutyl-4″-hydroxyphenyl)propionate]methane, octadecyl 3-(3″,5″-di-tert-butyl-4″-hydroxyphenyl)propionate, distearyl-pentaerythritol-diproprionate, thiodiethylene bis-(3,5-ter-butyl-4-hydroxy)hydrocinnamate, (1,3,5-trimethyl-2,4,6-tris[3,5-di-tert-butyl-4-hydroxybenzyl]benzene), 4,4″-methylenebis(2,6-di-tert-butylphenol), steraric acid, oleic acid, stearamide, behenamide, oleamide, erucamide, N,N″-ethylenebisstearamide, N,N″-ethylenebisoleamide, sterryl erucamide, erucyl erucamide, oleyl palmitamide, stearyl stearamide, erucyl stearamide, calcium sterate, other metal sterates, waxes (e.g. polyethylene, polypropylene, microcrystalline, carnauba, paraffin, montan, candelilla, beeswax, ozokerite, ceresine, and the like). The gel can also contain metallic pigments (aluminum and brass flakes), TiO2, mica, fluorescent dyes and pigments, phosphorescent pigments, aluminatrihydrate, antimony oxide, iron oxides (Fe304,-Fe203, etc.), iron cobalt oxides, chromium dioxide, iron, barium ferrite, strontium ferrite and other magnetic particle materials, molybdenum, silicone fluids, lake pigments, aluminates, ceramic pigments, ironblues, ultramarines, phthalocynines, azo pigments, carbon blacks, silicon dioxide, silica, clay, feldspar, glass microspheres, barium ferrite, wollastonite and the like.

Foam facilitators that are useful in the gel material include polyisobutylene, butyl rubber, surfactants, emulsifiers, dispersants and others.

Plasticizer bleed modifiers which tend to reduce plasticizer exudation from the gel material and which are useful therein include hydrocarbon resins, elastomeric diblock copolymers, polyisobutylene, butyl rubber, transpolyoctenylene rubber (tor rubber), and others.

Melt viscosity modifiers that tend to reduce the melt viscosity of the pre-compounded component mixture of the gel include hydrocarbon resins, transpolyoctenylene rubber, castor oil, linseed oil, non-ultra high molecular weight thermoplastic rubbers, surfactants, dispersants, emulsifiers, and others.

Melt viscosity modifiers that tend to increase the melt viscosity of the pre-compounded component mixture of the gel include hydrocarbon resins, butyl rubber, polyisobutylene, additional triblock copolymers having the general configuration A-B-A and a molecular weight greater than that of each of the principle elastomer components of the material, particulate fillers, microspheres, butadiene rubber, ethylene/propylene rubber, ethylene/butylene rubber, and others.

Tensile strength modifiers which tend to increase the tensile strength of the gel material include mid block B associating hydrocarbon resins, non-end block A solvating hydrocarbon resins, particulate reinforcers, and others.

Shrinkage inhibitors, which tend to reduce shrinkage of the material following compounding, that are useful in the present invention include hydrocarbon resins, particulate fillers, microspheres, transpolyoctenylene rubber, and others.

6. Microspheres

Microspheres may also be added to the gel to reduce specific gravity, save cost on elastomer, increase stiffness and achieve light weight. Different types of microspheres contribute various properties to the gel.

7. Physical Properties

When the preferred A-B-A triblock copolymer, plasticizer and additives are mixed, the resultant material is very strong, yet very elastic and easily stretched, having a Young's elasticity modulus of only up to about 1×10<6>dyne/cm**2 . The preferred gel also has low tack and little or no oil bleed and is capable of elongation up to about 1600% to 2400% and more.

8. EXAMPLES

The following examples are merely exemplary and suggestive of gel formulations which may be used in to make the surrogate sexual partner of the invention. The reader may choose any desired gel formulation when making a surrogate sexual partner and using the inventive concepts. Preferably, the gel formulation will be selected to achieve an artificial tissue having the resilience., hardness, and tactile feel of human flesh.

### EXAMPLE 1

Gelatinous Elastomer Having the Firmness of Muscle Tissue

| | A-B-A Triblock | | |
|---|---|---|---|
| | Copolymer | Plasticizer | Additives |
| Name | Kraton G1651 | Mineral Oil | Irganox 1010 |
| Amount | 100 parts | 500-1200 parts | 3 parts |
| | | (preferred 800 Parts) | |

### EXAMPLE 2

Gelatinous Elastomer Having the Firmness of Fatty Tissue

| | A-B-A Triblock | | |
|---|---|---|---|
| | Copolymer | Plasticizer | Additives |
| Name | Kraton G1651 | Mineral Oil | Irganox 1010 |
| Amount | 100 parts | 1100-1600 parts | 3 parts |
| | | (preferred 1400 Parts) | |

### EXAMPLE 3

Gelatinous Elastomer Having the Firmness of Mucous Membranes

| | A-B-A Triblock | | |
|---|---|---|---|
| | Copolymer | Plasticizer | Additives |
| Name | Kraton G1651 | Mineral Oil | Irganox 1010 |
| Amount | 100 parts | 1100-1600 parts | 3 parts |
| | | (preferred 1400 Parts) | |

The structures used in the articulating skeleton of the surrogate sexual partner are described under heading “C” below.

B. Manufacturing Methods

In order to manufacture a surrogate sexual partner of the invention, the first step is to make gelatinous elastomers of the desired hardness and consistency for the various body parts to be made. Generally, different forumations of gelatinous elastomer will be required for three different areas of the surrogate sexual partner: (i) mucous membrane areas such as vagina, anal cavity and mouth, (ii) fatty tissue areas, and (iii) muscle areas. The gelatinous elastomers are made by compounding an A-B-A triblock copolymer with a plasticizing agent.

1. Melt Blending

A preferred method for making suitable gelatinous elastomers for use in the present invention includes mixing the plasticizer, triblock copolymer and any additives and/or microspheres, heating the mixture to melting while agitating the mixture, and cooling the compound. This process is referred to as “melt blending.”

Excessive heat is known to cause the degradation of the elastomeric B portion of A-B-A and A-B block copolymers. Similarly, maintaining block copolymers at increased temperatures over prolonged periods of time often results in the degradation of the elastomeric B portion of A-B-A and A-B block copolymers. As the B molecules of an A-B-A triblock copolymer break, the triblock is separated into two diblock copolymers having the general configuration A-B. While it is believed by some in the art that the presence of A-B diblock copolymers in oil-containing plasticizer-extended A-B-A triblock copolymers reduces plasticizer bleed-out, high amounts of A-B copolymers significantly reduce the strength of the material of the present invention. Thus, Applicant believes that it is important to minimize the compounding temperatures and the amount of time to which the material is exposed to heat.

The plasticizer(s), any additives and/or microspheres, and the A-B-A copolymers are premixed. Preferably, hydrophobic additives are dissolved into the plasticizer prior to adding the plasticizer to the elastomer component. Hydrophilic additives and particulate additives are preferably emulsified or mixed into the plasticizer of a preferred material of the present invention prior to adding the elastomer component. The mixture is then quickly heated to melting. Preferably, the temperature of the mixture does not exceed the volatilization temperature of any component. For most of the materials of the invention, Applicant prefers temperatures in the range of about 260 degrees F. to about 290 degrees F. A melting time of about ten minutes or less is preferred. A melting time of about five minutes or less is more preferred. Even more preferred are melting times of about ninety seconds or less. Stirring, agitation, or, most preferably, high shearing forces are preferred to create a homogeneous mixture (e.g., as with a planetary mixer or twin-screw injector). The mixture may then cast, extruded, injection molded, etc. Alternatively, next, the mixture is cooled.

Use of high shear facilitates short heating times. “High shear”, for purposes of this disclosure, is defined in terms of the length over diameter (L/D) ratio of a properly designed injection molding single screw or extruder single screw. L/D ratios of about 20:1 and higher create high shear. Twin screws, Banbury or planetary mixers and the like also create high shear. High shearing with heat mixes compounds at lower temperatures and faster rates than the use of heat alone or heat with relatively low-shear mixing. Thus, high shear forces expedite compounding of the mixture over a relatively short period of time by more readily forcing the molecules into close association with the B component of the A-B-A copolymer. Use of high shear also facilitates the decrease of equipment temperatures. Melt blending techniques which employ little or no shear require an external heat source. Thus, in order to avoid heat loss, the periphery of many types of melt blending equipment must be heated to a temperature higher than the melt temperature in order to transfer heat and melt a component mixture. In comparison, high shearing equipment can generate high material temperatures directly from the shear forces, substantially reducing or eliminating the need for external heating.

The inventor prefers the use of equipment that produces high shear, such as twin screw compounding extrusion machinery, to melt blend the material of the present invention. Twin screw compounding extrusion machinery is desired for compounding the preferred materials of the present invention since it generates a very high level of shear and because compounding and molding, casting, extrusion, or foaming are performed in one continuous process. Alternatively, the elastomeric gel may be compounded first, then later formed into a finished product by casting, injection molding, extrusion, or some other method.

Microspheres may be added to the gel to reduce its specific gravity, to increase its stiffness or durometer, and to increase its rebound rate. Glass microspheres usually will not survive high shear. However, acrylic microspheres remain intact when subjected to the heat and shear of injection molding machines and extruders if the time at high temperature is kept to about five minutes or less. Thus, acrylic microspheres are preferred over glass microspheres.

Other equipment, such as batch mixers are also useful for melt blending the preferred materials of the present invention.

The compositions of the present invention are prepared by blending together the components including other additives as desired at about 23 degrees C. to about 100 degrees C. forming a paste like mixture and further heating said mixture uniformly to about 150 degrees C. to about 200 degree C. until a homogeneous molten blend is obtained. Lower and higher temperatures can also be utilized depending on the viscosity of the oils and amount of SEBS used. These components blend easily in the melt and a heated vessel equipped with a stirrer is all that is required. As an example, small batches can be easily blended in a test tube using a glass stirring rod for mixing. While conventional large vessels with pressure and/or vacuum means can be utilized in forming large batches of the instant compositions in amounts of about 40 lbs or less to 10,000 lbs or more. For example, in a large vessel, inert gases can be employed for removing the composition from a closed vessel at the end of mixing and a partial vacuum can be applied to remove any entrapped bubbles. Stirring rates can be tailored to the user's particular needs, utilized for large batches can range from about less than about 10 rpm to about 40 rpm or higher.

Especially suitable gels can be prepared by melt blending an admixture comprising: (A) 100 parts by weight of a high viscosity triblock copolymer of the general configurations poly(styrene-ethylene-butylene-styrene) or poly(styrene-ethylene-propylene-styrene) (herein referred to as “SEBS” or “SEPS”) where said triblock copolymer is characterized as having a Brookfield Viscosity of a 20 weight percent solids solution of said triblock copolymer in toluene at 25o C. of about 1,800 cps and higher. (B) from about 200 to about 1,300 parts by weight of an plasticizing oil.

While preferred components and formulation ranges have been disclosed herein, persons of skill in the art can extend these ranges using appropriate material according to the principles discussed herein. All such variations and deviations which rely on the teachings through which the present invention has advanced the art are considered to be within the spirit and scope of the present invention.

2. Solvent Blending

A second method for making the gelastinous elastomers used in the present invention involve dissolving the elastomeric component in a solvent, adding plasticizer and any additives and/or microspheres, and removing the solvent from the mixture.

Aromatic hydrocarbon solvents such as toluene may be used for mixing the preferred compounds of the present invention. Sufficient solvent is added to the elastomer component to dissolve the network of block copolymer molecules. Preferably, the amount of solvent is limited to an amount sufficient for dissolving the network of block copolymer molecules. The elastomer then dissolves in the solvent. Mixing is preferred since it speeds up the solvation process. Similarly, slightly elevating the mixture temperature is preferred since it speeds up the solvation process. Next, plasticizer and additives are mixed into the solvated elastomer. Hydrophobic additives are preferably dissolved in the plasticizer prior to adding the plasticizer to the elastomer component and the solvent. Hydrophilic additives and particulate additives are preferably emulsified or mixed into the plasticizer prior to adding the elastomer component and solvent. The mixture is then cast into a desired shape (accounting for later shrinkage due to solvent loss) and the solvent is evaporated from the mixture.

3. Foaming

It is possible to prepare the gelatinous elastomer used in the present invention and even to make the surrogate sexual partner itself by foaming. “Foaming”, as defined herein, refers to processes which form gas bubbles or gas pockets in the gelatinous elastomer.

Various methods for foaming the gel include, but are not limited to whipping or injecting air bubbles into the material while it is in a molten state, adding compressed gas or air to the material while it is in the molten state and under pressure, adding water to the material while it is in the molten state, use of sodium bicarbonate, and use of chemical blowing agents.

4. Shaping

After desired gelatinous elastomers have been prepared for making the surrogate sexual partner, shaping of the gelatinous elastomers into the desired configuration can begin. First, it is necessary to obtain a form or mold of the desired shape in which to form the surrogate sexual partner. The form or mold should be of the desired body proportions of the finished surrogate sexual partner. The form or mold can be of an appropriate material, such as metal, wood, plastic, or other materials. It should be noted that if it is intended for the surrogate sexual partner to have desired physical characteristics, such as a celebrity likeness or particular body proportions, a custom mold must be created for that particular application.

A preferred method for obtaining a custom mold is the process of life-casting, whereby the model's body is covered with fast-curing, non-toxic RTV silicone, such as Ply-o-Life, available from Pink House Studio in St. Albans, Vt. or Dermasil from Douglas and Sturgess of San Francisco, Calif. Then a temporary mother mold of plaster bandages is applied in two halves (front and back) to the outside surface of the silicone. After hardening, the mother mold halves are removed and the silicone is cut and peeled from the model, taking care to leave a clean seam that will easily mate later on. For the model's comfort it is necessary to make 3 separate molds: of 1) the torso, legs, and feet, 2) the arms and hands, and 3) the head. Once the pieces of the mold have cured they are re-assembled and cast into.

A preferred method of casting is with a hand lay up using Forton MG (an epoxy-like gypsum product) and biaxial fiberglass. The Forton MG Casting System is odorless and easy to use for solid and hollow castings. The procedure is essentially like that used for casting in polyester resin and fiberglass. Forton is easier to work with, so a casting made with this material takes less time to complete. Furthermore, patching, sanding and finishing are all easier on a casting made of Forton than on a casting made of polyester resin or epoxy. An additional advantage is that, unlike polyester and epoxy resins, Forton may be cast directly into an alginate mold. From the standpoint of health and safety the Forton casting system is considerably less hazardous when compared with polyester resin or epoxy casting.

Like the mold itself, the lay up (casting) is done in pieces—arms, head, and torso. Then the pieces are assembled (grinding as necessary to make them fit together). Pin holes and other imperfections are filled, and the casting is prepared for the process of creating the production mold.

Since this part of the process does not involve a living model, a stronger, long-curing RTV silicone can be used, such as PlatSil 73 from PolyTech Development Corp. of Laston, Pa. The PlatSil® 73 Series RTV silicone rubbers are two-component, addition-cure, platinum-catalyzed, very high tear strength, flexible mold compounds suitable for a production mold.

After the silicon is cured, a production mother mold is created (again in 2 halves, front and back), with a hole in the top of the head into which the molten Kraton will be poured. The preferred material for the mother mold is Forton MG from Pink House or Poly 15-3 from PolyTech.

Once the mold has been selected, the skeletal structure of the surrogate sexual partner should be arranged within the mold. The skeletal structure may be held in place with pins, bolts, wires or other fixtures so that it will be in the appropriate position in the finished surrogate sexual partner.

After the skeletal structure is in place in the mold, the gelatinous elastomer is introduced. This may be achieved by simple pouring and casting, injection molding, spraying, foaming or otherwise. Preferably, firm gelatinous elastomer resembling muscle tissue in consistency will be shaped for the majority of the body of the surrogate sexual partner first. Inserts into the mold are used to create orifices during shaping. After shaping of the firm gelatinous elastomer, soft gelatinous elastomer representing Kraton may be formed in areas where fatty tissue would be present (breasts, buttocks, tummy, etc.) in order to create a realistic feel. Next, the lining of orifices such as mouth, anal cavity and vagina will be coated with a gelatinous elastomer formulation having a feel similar to the mucous membranes of these body parts.

Best bonding of soft elastomeric gel body portions is achieved by casting them simultaneously. This is achieved by first casting the firm gel into the mold (almost up to the neck). Then a long, narrow injection tube is inserted into the mold through the hole in the top of the head until it reaches alternately the vagina, rectum, and breasts. The soft gel is injected through the tube, displacing the still molten firm gel in those specific parts. It may be helpful to use separate tubes for each soft part to be cast so that the tubes can be precisely secured in place. They are withdrawn from the mold while the material is still molten.

Finally, finishing is performed. This will include the attachment of hair to the head and pubic areas as desired, the attachment of hair to serve as eyebrows and eyelashes, the insertion of realistic glass eyes, the painting or dying of lips, the attachment of fingernails, and any other desired finishing operation. An internal voice chip may be included in the surrogate sexual partner in order to produce audio realism.

Referring to FIG. 15, a headset assembly that may be used with the surrogate sexual partner is depicted 1501. It may be placed on a base 1502 when not in use. A preferred audio system for the surrogate sexual partner includes an MP3 player such as the RIO 300, available from S3 Incorporated (formerly Diamond Multimedia Systems) of Santa Clara, Calif. and an infrared wireless headset such as the Model 60001R from UnWired of Farmingdale, N.Y. The advantage of the Rio 300 is that it stores up to an hour of digital quality recording in MP3 compression format that can be bookmarked and accessed randomly (without fast-forwarding as with cassette tapes), and favorite selections can be looped. The user can also customize the sound track with his own recorded content. The player can be housed in a receptacle in the sexual surrogate partner's head, or it can be kept externally (near enough to reset, but out of the way). The advantage of the wireless headphones is that they deliver high-fidelity sound without the disadvantage of a tangled cord. The sound is much more realistic when delivered through headphones (as opposed to external speakers), seaming to surround and envelope the user, while assuring him of privacy. The track itself is the voice of the celebrity model as recorded during sex. In order to make the audio portion of the experience as realistic and compelling as possible, there is little talking, mostly breathing, moans, the soft slapping of flesh against flesh, and other appropriate sounds. A sound system is provided in order to provide the user with auditory stimulation.

C. Components of One Preferred Embodiment

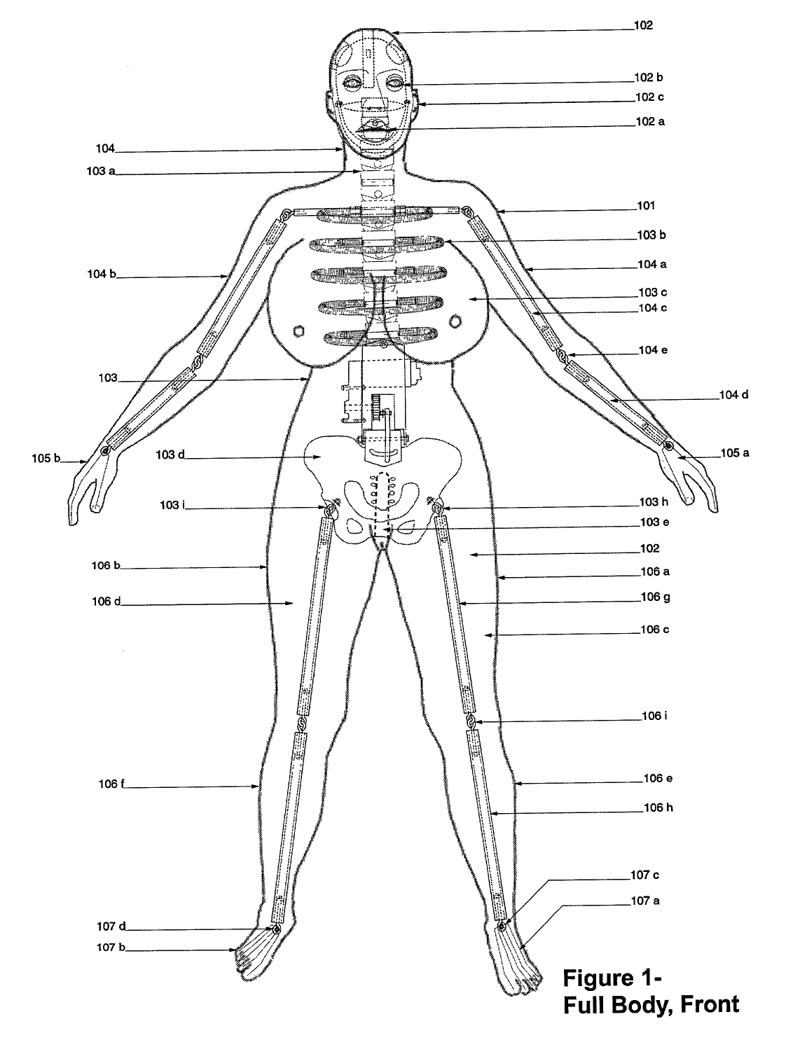

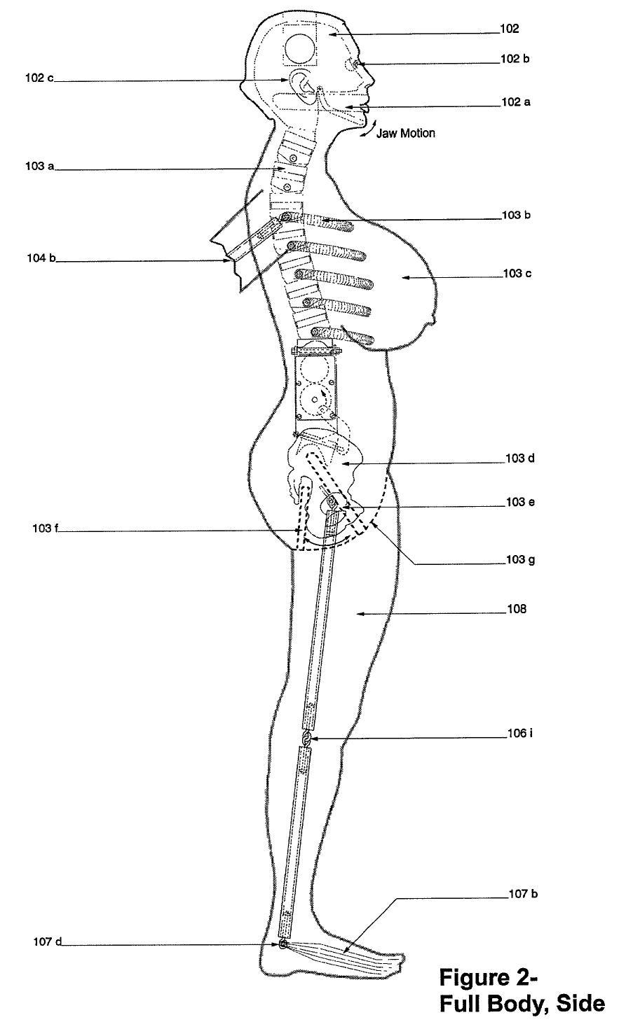

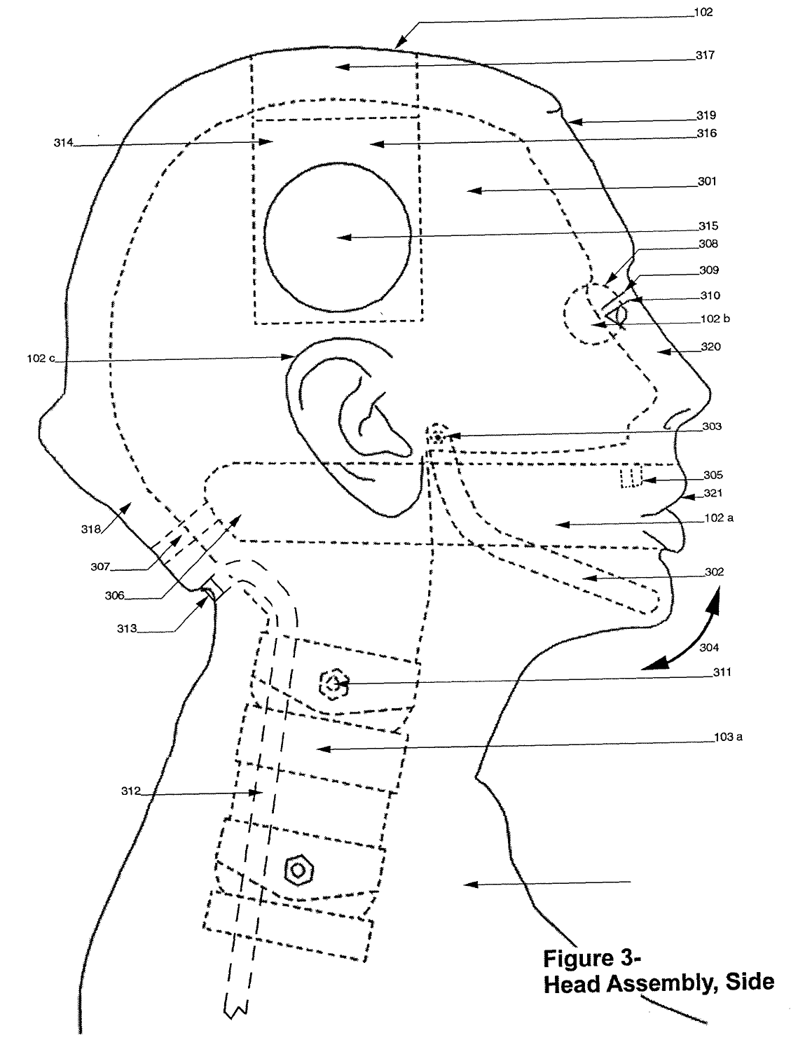

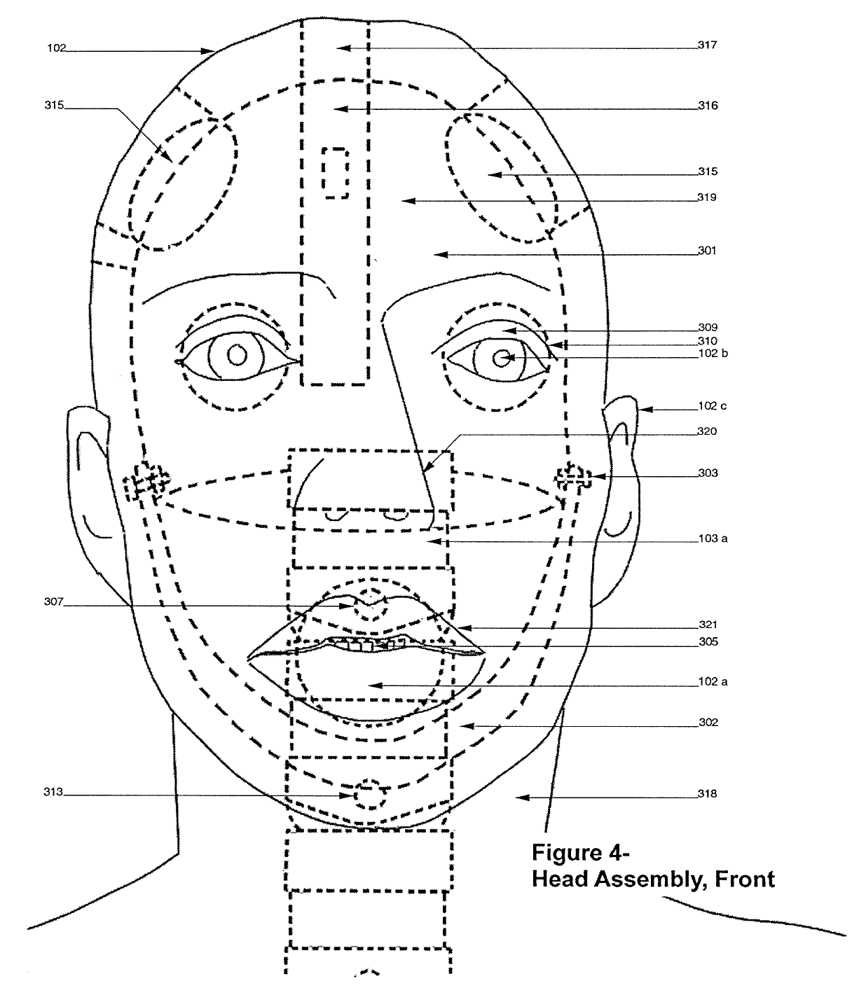

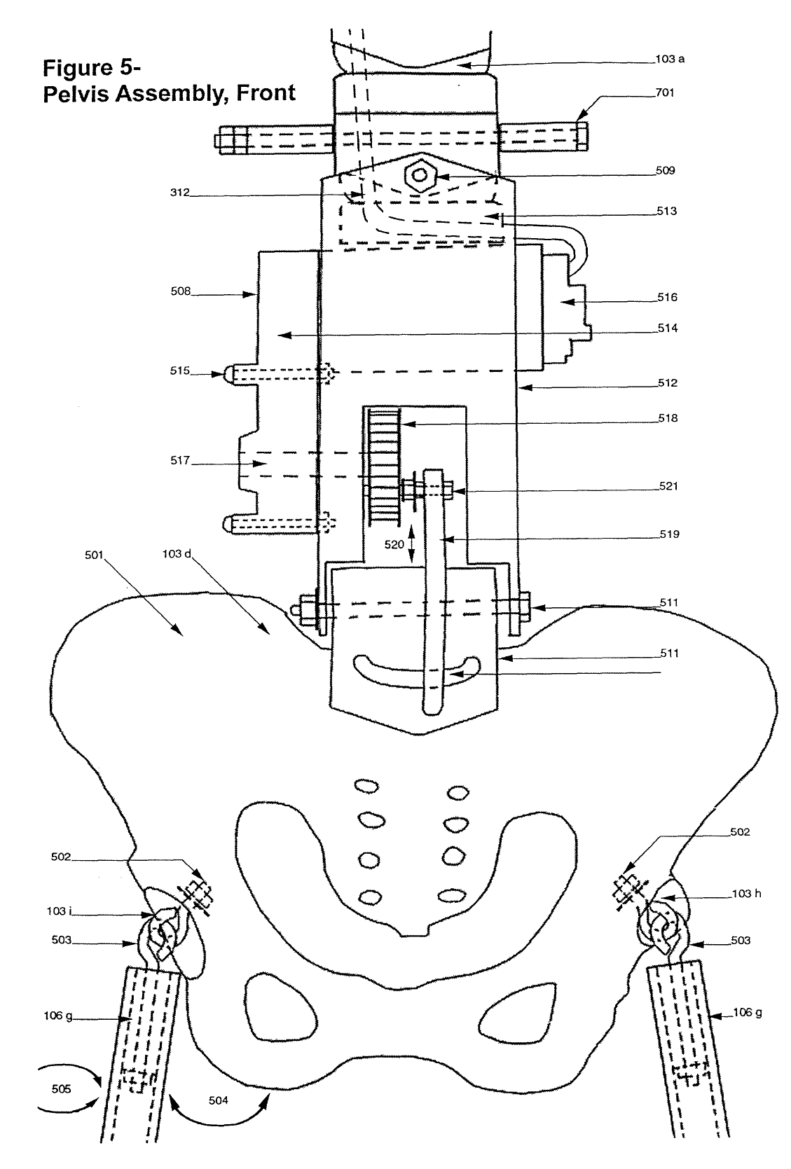

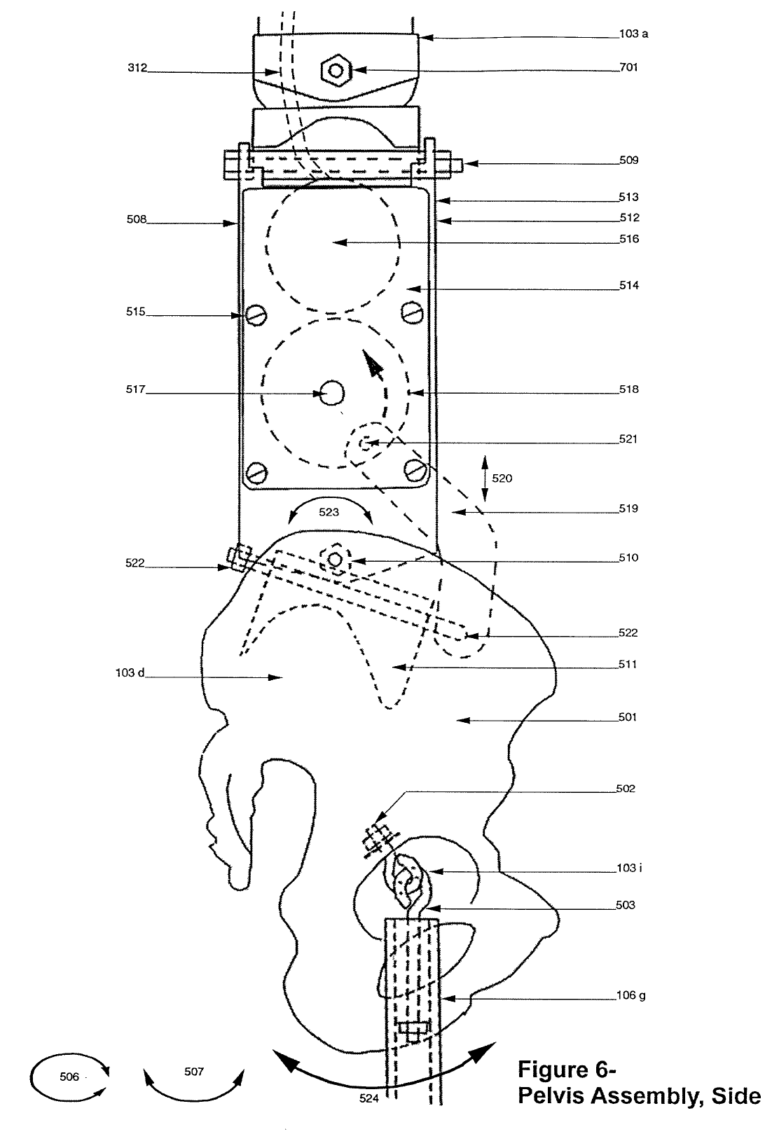

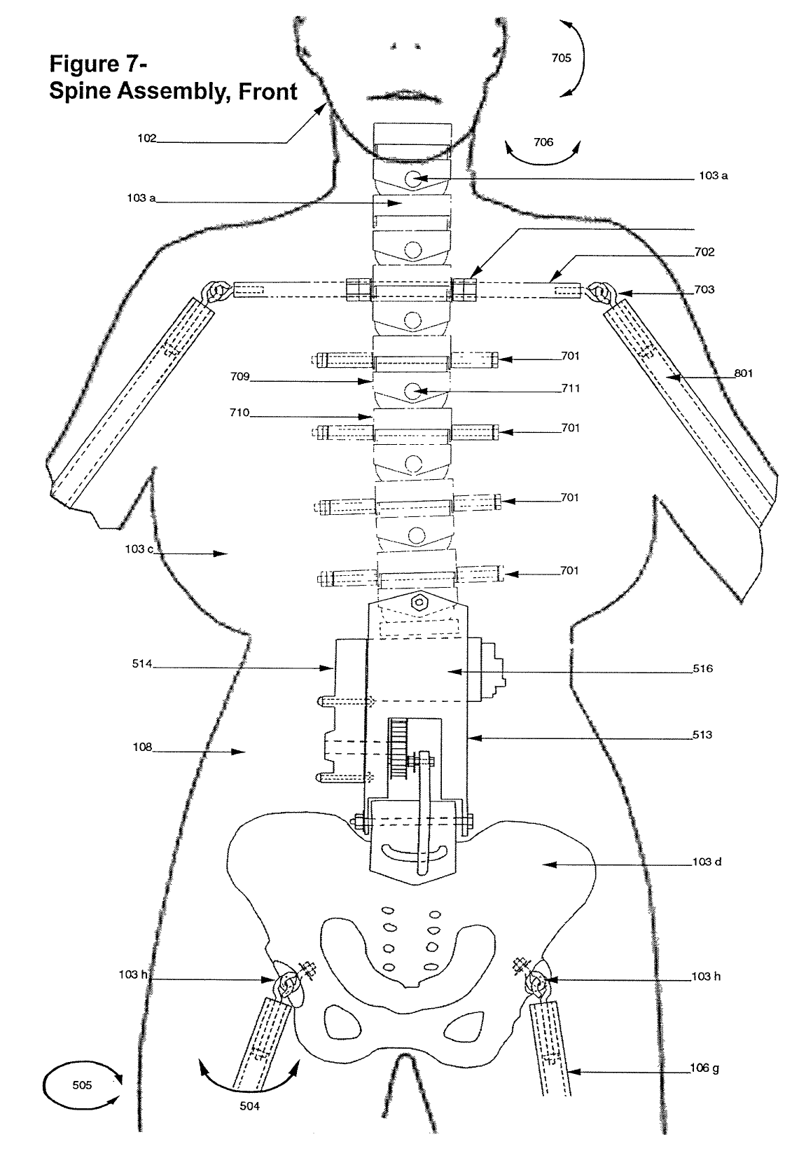

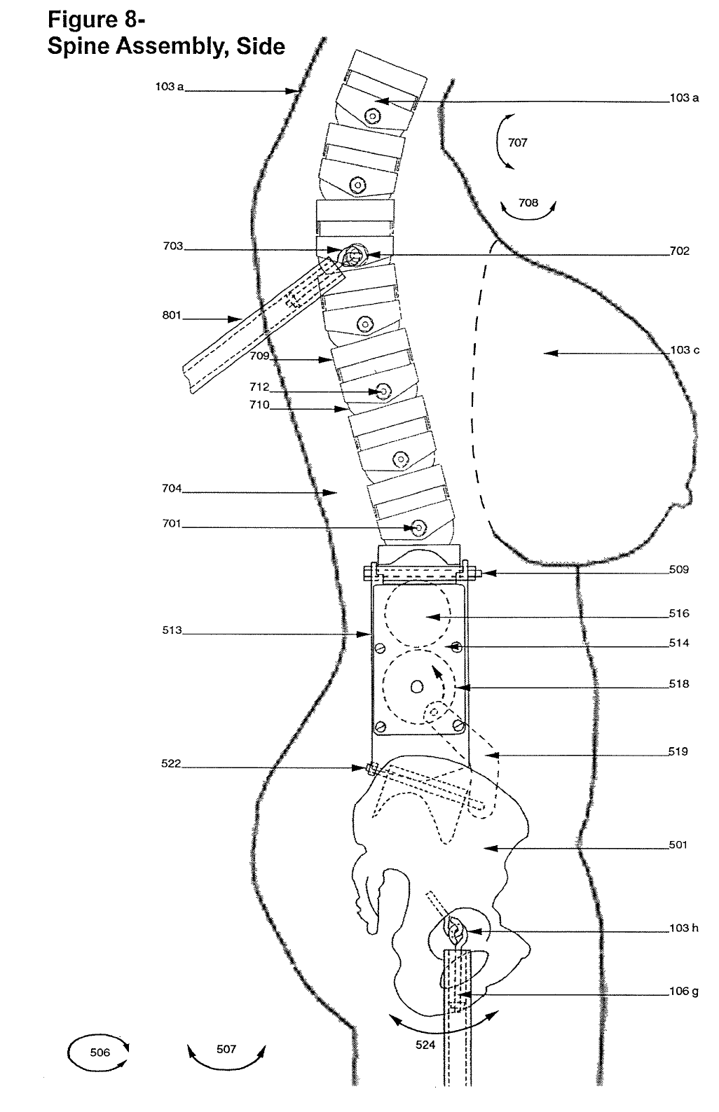

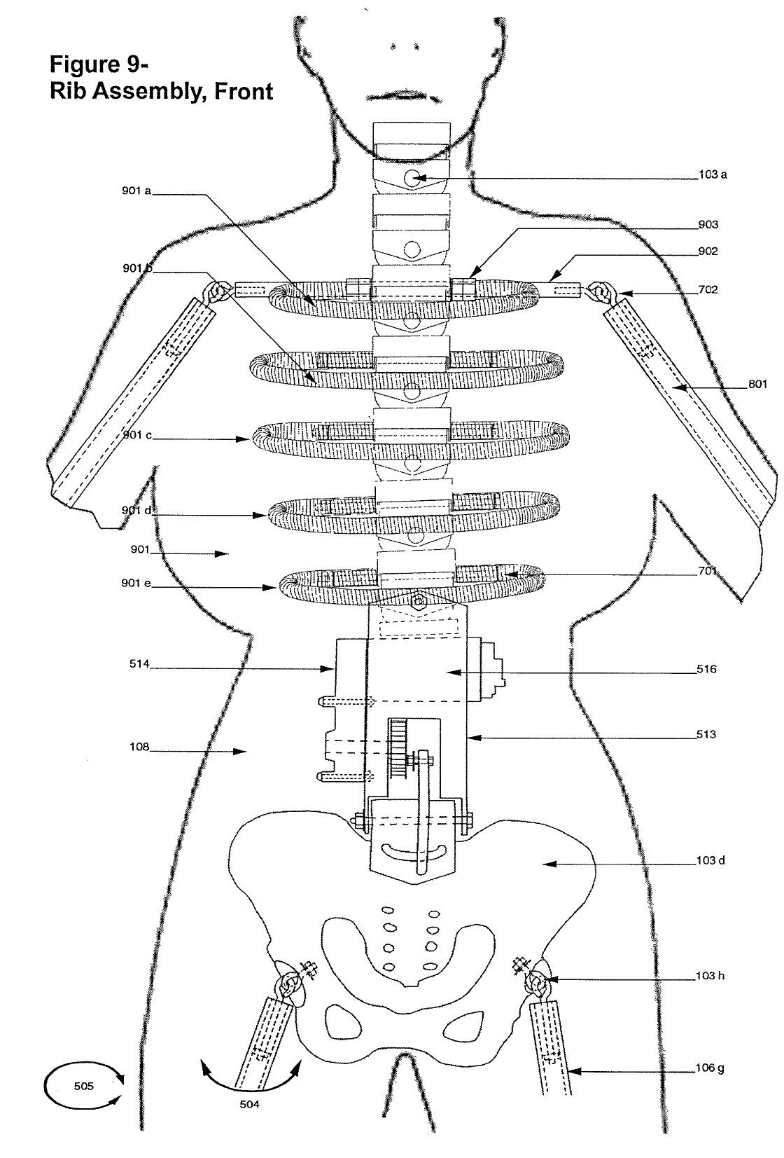

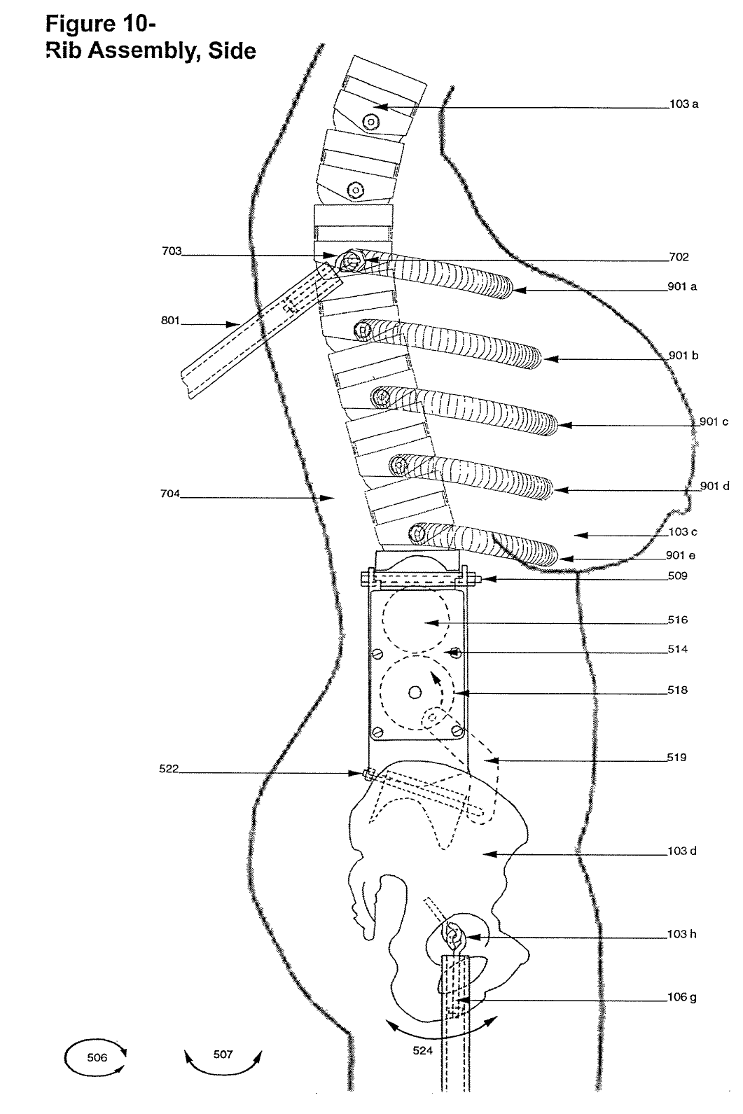

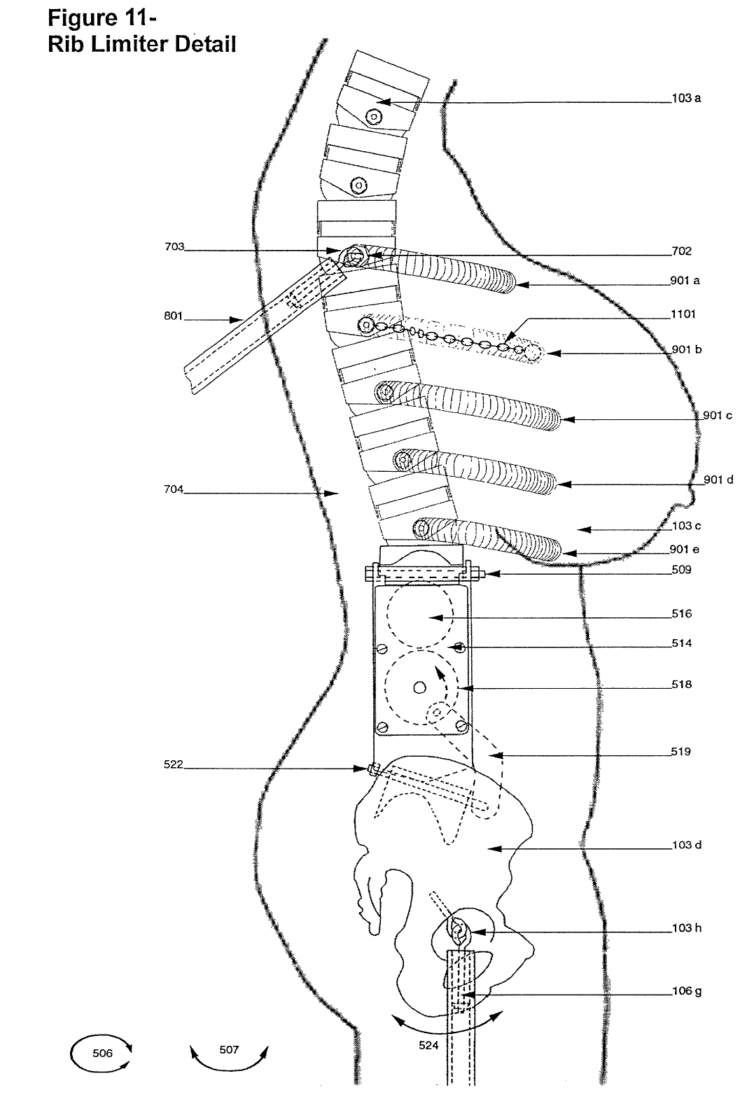

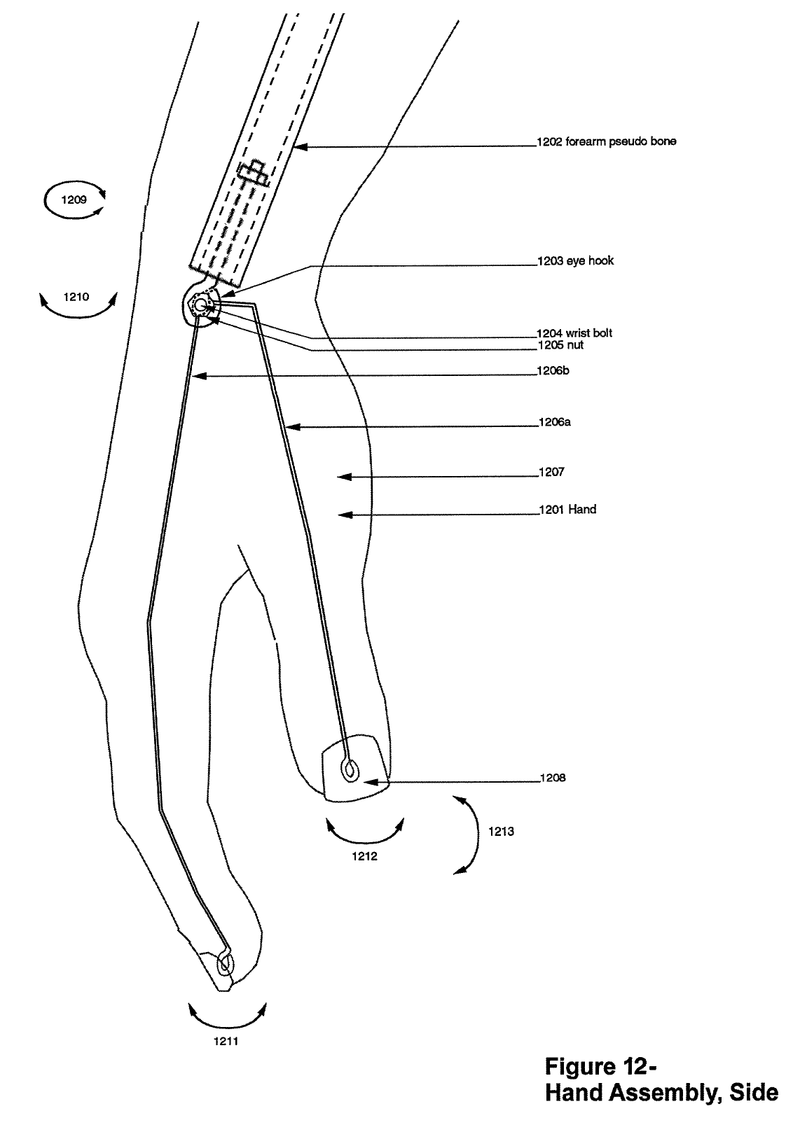

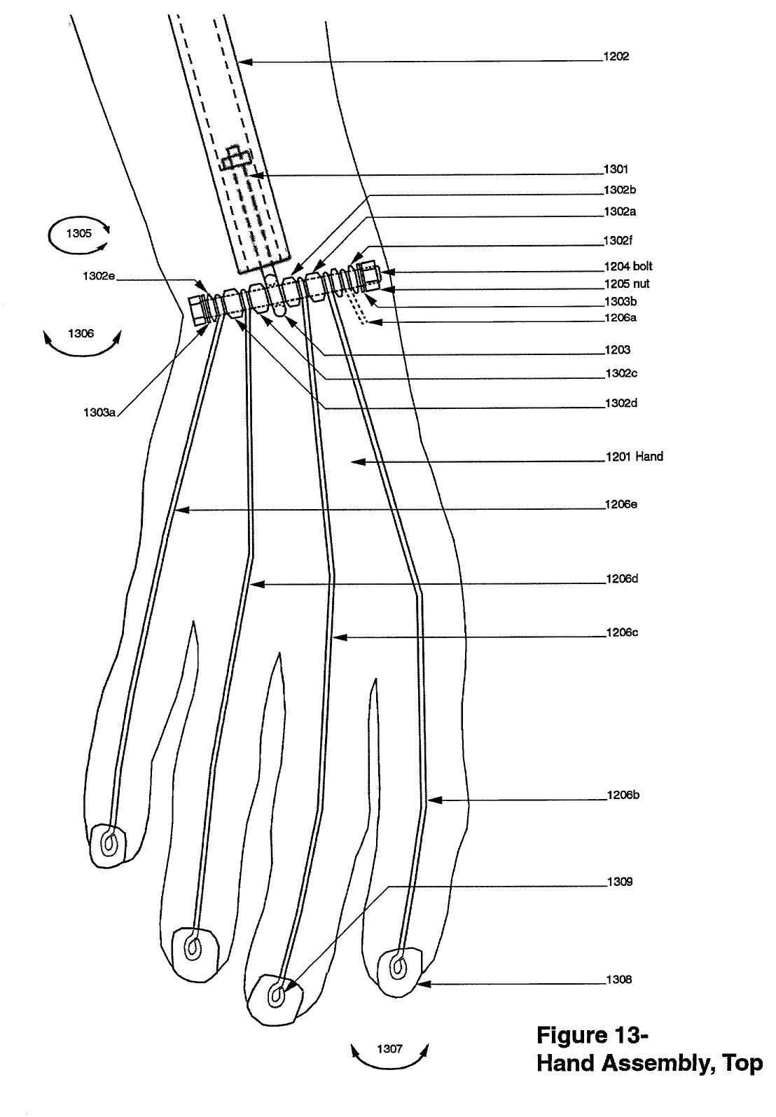

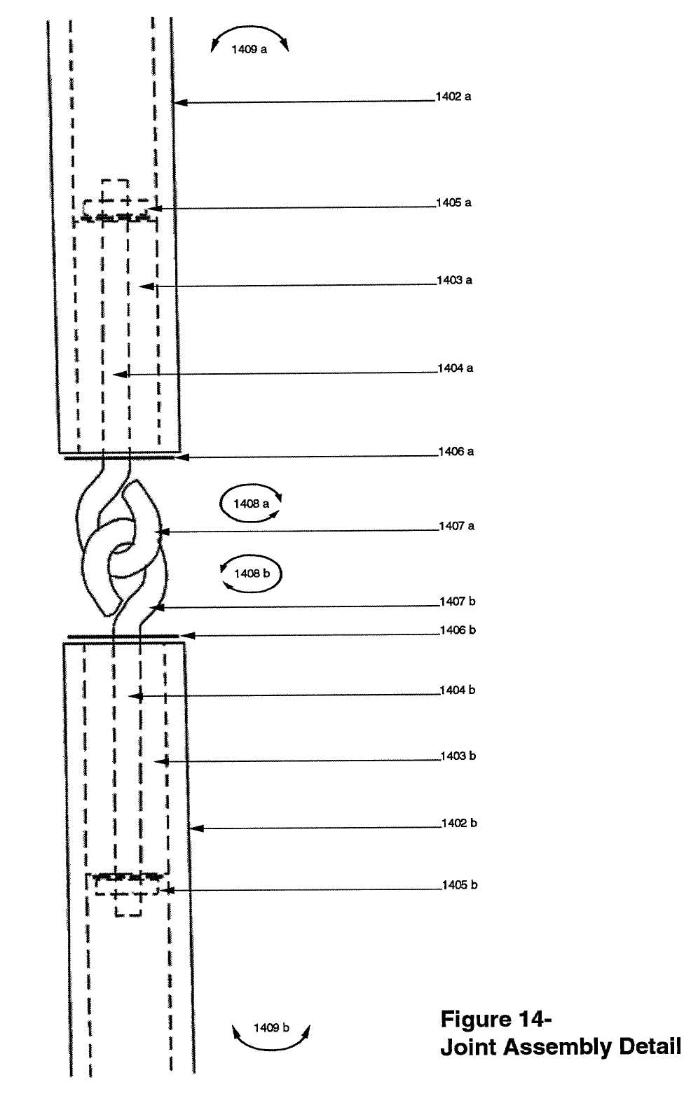

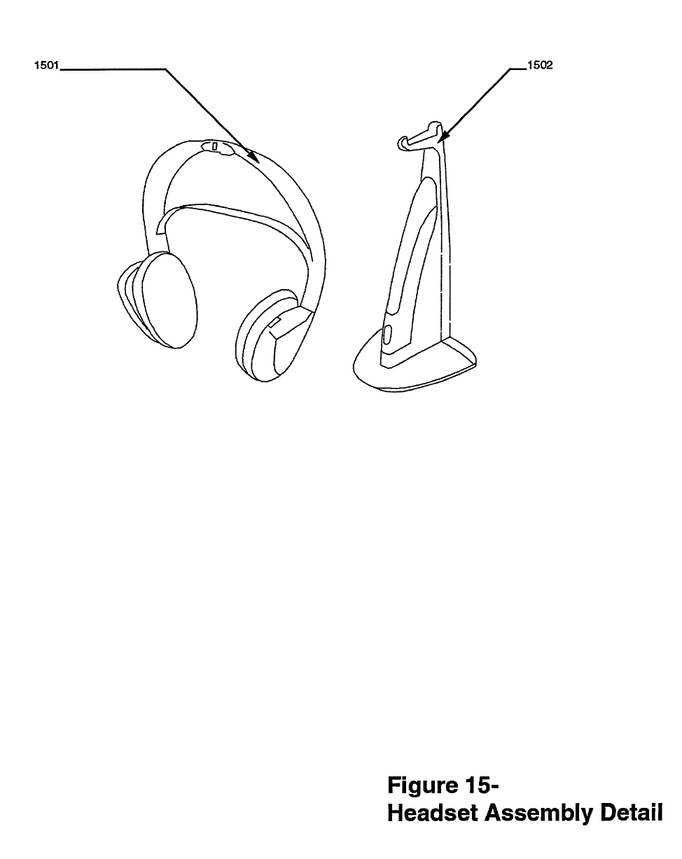

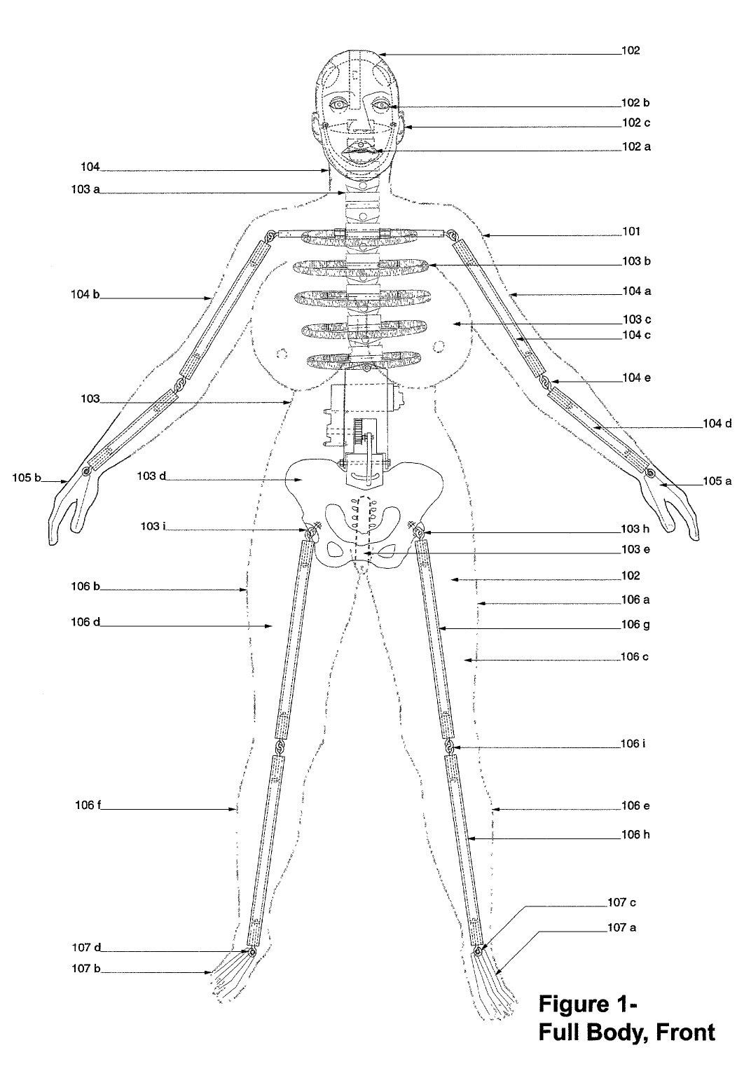

Referring to FIG. 1, one preferred embodiment of a surrogate sexual partner 101 of the invention is depicted. As an introductory matter, the reader should be aware that the entirety of the surrogate sexual partner 101 is constructed to be of lifelike size, proportion, color and feel in order to far more closely approximate the presence of a real human than prior art devices.

The surrogate sexual partner 101 includes a number of components. A head assembly 102 is provided. The head assembly 102 includes oral cavity 102a, eyes 102b, ears 102c and other structures to cause the head assembly 102 to have a realistic appearance.

The head assembly 102 is attached to a torso 103 by a neck 104. The torso 103 includes features such as an elongate spine 103a, a rib assembly 103b, breasts 103c and a functional pelvis 103d. In the vicinity of the pelvis 103d, a vaginal cavity 103e is located. A left arm 104a and a right arm 104b are attached to the torso 103. Each arm 104a and 104b has an upper arm pseudo-bone 104c, a lower arm pseudo bone 104d, and a joint assembly 104e formed by two eye bolts Each arm 104a and 104b has a hand 105a and 105b attached to it. Also attached to the torso 103 in the vicinity of the pelvis assembly 103d, two legs 106a and 106b are located. The legs 106a and 106b are attached to the pelvis 103d by a double eye bolt hip joint 103h and 103i. Each leg has a thigh 106c and 106d and a calf 106e and 106f. Each leg 106a and 106b has located within it an upper leg pseudo-bone 106g and a lower leg pseudo-bone 106h, connected at a knee joint 106i by two eye bolts. About the pseudo bone, muscle tissue is present. Feet 107a and 107b are provided. The feet 107a and 107b are attached to lower leg pseudo bone by a single eye bolt ankle joint 107c and 107d.

An alternate embodiment of a joint is a universal joint. The advantage of the universal joint is that it allows the limbs to articulate with many degrees of freedom. If the joints are tightened slightly, the friction can make the sexual surrogate partner's limbs more poseable (able to maintain a set position until moved). The chief advantage of the two eye bolt approach is that it allows the same range of motion at a much lower cost (about 6% of the cost of universal joints available from W. W. Grainger, Incorporated of Lake Forrest, Ill.).

Flesh portions of the surrogate sexual partner can be classified generally as replicating muscle tissue (upper and lower arms, torso, thighs, calves, neck), fatty tissue (tummy, buttocks, breasts), and mucous membranes (oral cavity, vaginal cavity, anal cavity). These are referred to as muscle tissue gelatinous elastomer, fatty tissue gelatinous elastomer and mucous membrane gelatinous elastomer. Examples of such gelatinous elastomers are provided above. Approximate firmness of these gelatinous elastomers is as follows:

| | HARDNESS/FIRMNESS |
|---|---|
| GELATINOUS ELASTOMER | (SHORE A SCALE) |
| Muscle Tissue Gelatinous Elastomer | 85-350 |
| Fatty Tissue Gelatinous Elastomer | 50-93 |
| Mucous Membranes Gelatinous Elastomer | 50-93 |

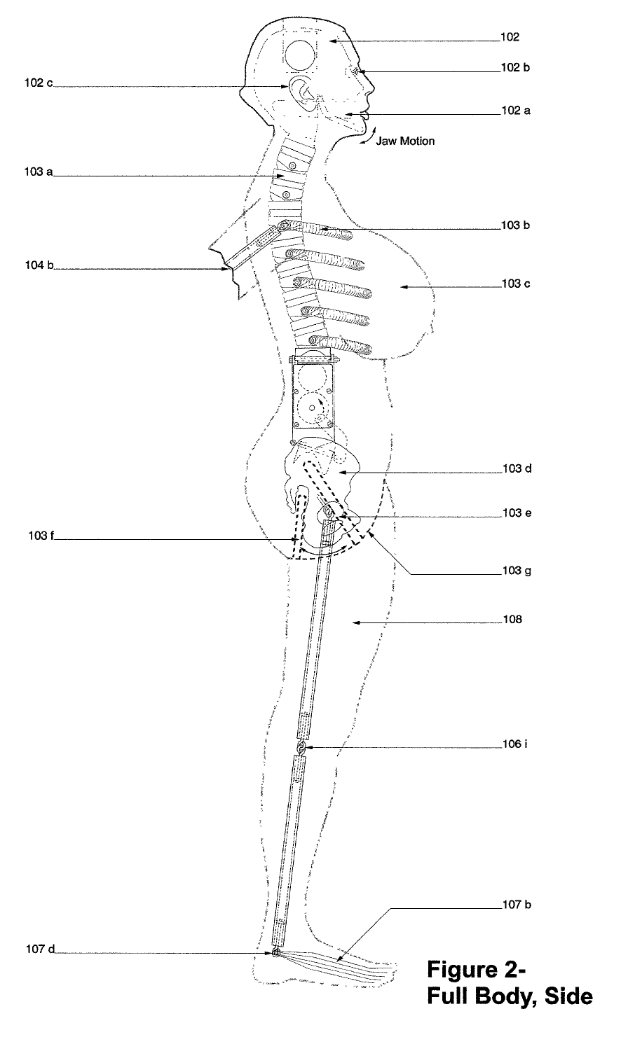

Referring to FIG. 2, a full body side view of a surrogate sexual partner 101 of the invention is depicted. The surrogate sexual partner 101 depicted includes those features described above. It also includes a rectal cavity 103f and optional pubic hair 103g. The rib assembly as shown includes a plurality of ribs, in this instance five ribs. However, the rib assembly could include any desired number of ribs, or a unitary network structure in its place. The advantage of using springs in the rib assemblies is that they can withstand the heat of casting without deformation. At the same time they help to increase shape memory of the torso (the part of the sexual surrogate partner that has the largest cross-sectional area), which would otherwise tend to flatten out unrealistically under the weight of the breasts. Without the spring ribs, a firmer formulation of gel would need to be used. With the addition of springs, the realism of upper body bending and rotating is increased.

In the invented surrogate sexual partner devices, the hair for pubic hear, hair for the head assembly, hair for eyebrows and hair for eyelashes may be artificial hair such as nylon, or genuine human hair. It is anticipated that genuine human hair will be used on premium models for optimum realism.

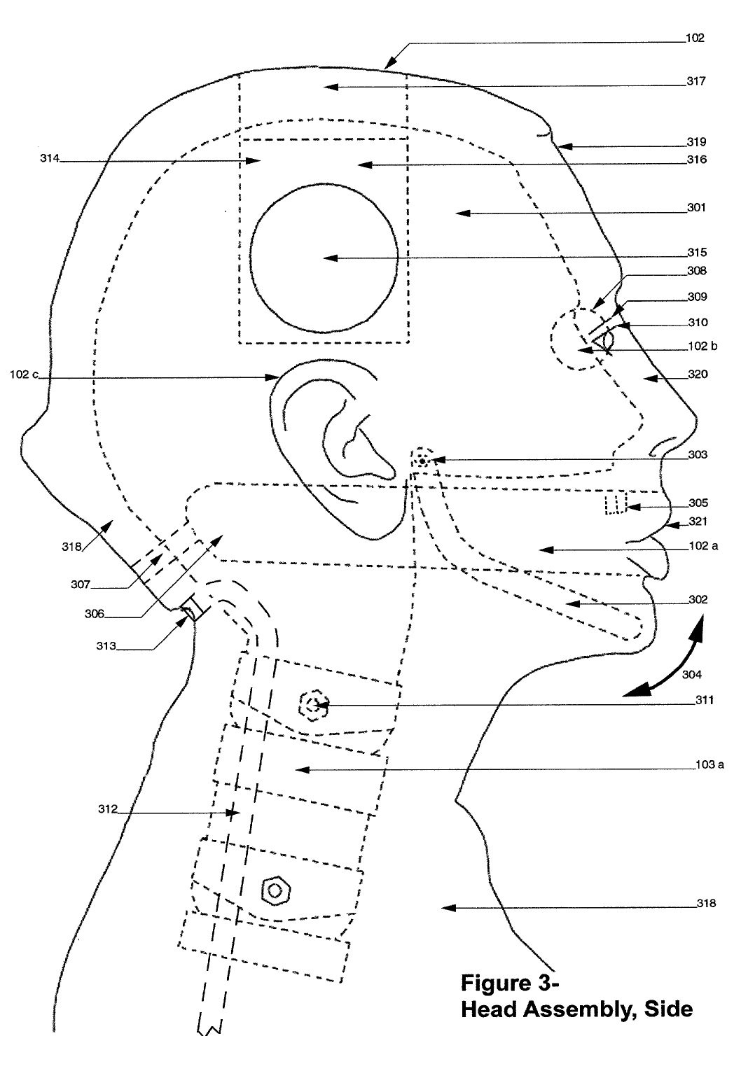

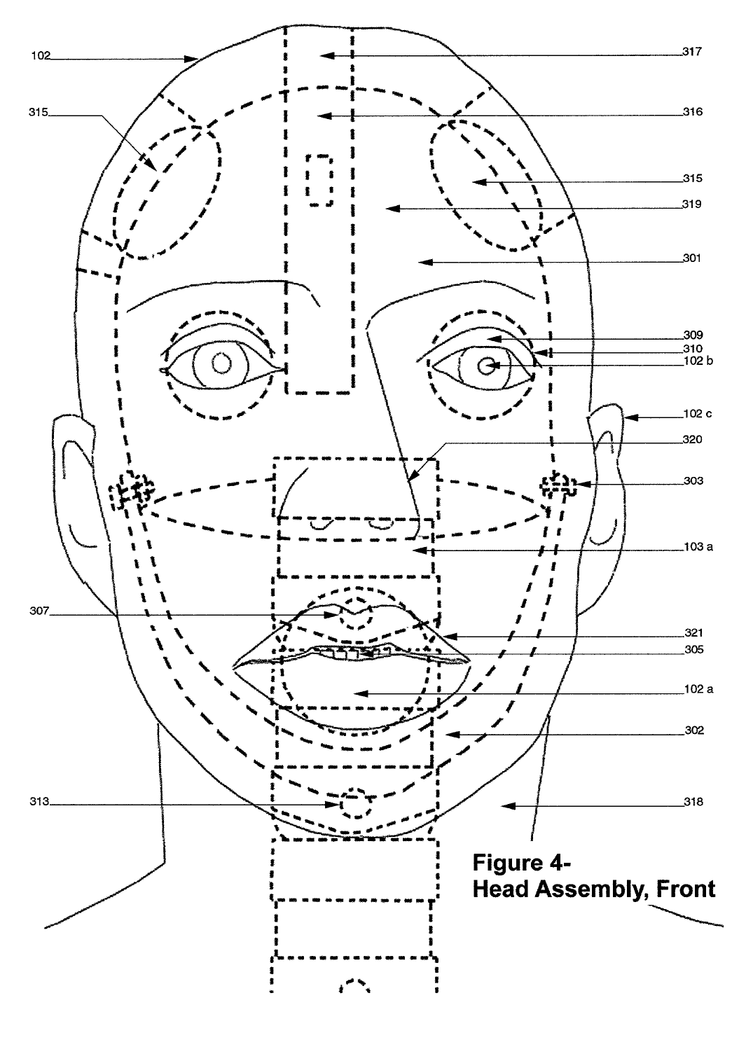

Referring to FIGS. 3 and 4, side view and a front view of a head assembly 102 of one embodiment of a surrogate sexual partner is depicted. The head assembly 102 includes an ear 102c formed from firm gelatinous elastomer in the approximate shape of a human ear. An oral cavity 102a is provided that is lined with a soft gelatinous elastomer that approximates the feel of a mucous membrane. The oral cavity 102a is formed between the lower portion of the pseudo-skull 301 and the upper part of the jaw bone 302. The pseudo-skull 301 is formed from a rigid material such as plastic, cured resin, epoxy, ceramic or other suitable materials, in the approximate shape of a human skull. The jaw pseudo-bone 302 is formed from a rigid material such as a metal or aluminum, and extends in dog-leg or curvilinear fashion from a jaw pivot 303 on the pseudo-skull 301. The jaw pivot 303 permits the jaw pseudo-bone 302 to pivot with respect to the pseudo-skull 301 as depicted by arrow 304. Teeth 305 formed from an appropriate material such as a firm gelatinous elastomer may be provided in the oral cavity 102a. The oral cavity 102a extends into the head assembly 102 to a distal end 306. Optionally, a removable tongue (not shown) may be included for realism if the user prefers French kissing or oral sex. Preferably the distal end 306 of the oral cavity 102a has a vent hole 307 for gaseous communication with the atmosphere and for cleaning purposes. If a suction effect is desired, the vent hole may be temporarily blocked as desired. An alternative embodiment may provide an internal chamber or bladder at the end of the vent hole (inside the head) into which the trapped air from the oral cavity may be forced as the cavity is filled. This avoids the disadvantage of unrealistic facial distortion during use. The oral cavity 102a is sized and shaped to accommodate insertion of a male sexual organ therein, and the vent hole 307 avoids creating of unwanted suction with a male sex organ.

An eye socket 308 is formed in the pseudo-skull 301 in order to accommodate a prosthetic eye 102b therein for realism. Optional eyebrows 309 and eyelashes 310 may be included as well.

The pseudo skull 301 is connected at its lower extremity to a spine 103a by an appropriate connector such as a nut and bolt assembly 311. The head assembly can articulate with respect to the spine at the location where the spine and skull connect, in order to provide lifelike movement. Through the spine 103a and exiting at the base of the pseudo skull 301 is a power cord 312 having a plug or fixture 313 for receiving electrical power. Electrical power is transported by the power cord 312 to a motor 516, and the motor gear winding which provides pelvic motion.

The pseudo skull 301 has a receptacle 314 in an appropriate location. The receptacle 314 is formed to receive a sound producing device 316 which may include a speaker and foam plug 315. A skull plug 317 may be used to conceal the sound producing device 316 and keep it in place. The sound producing device 316 may include two speakers for stereo sound as shown in FIG. 4. The sound producing device may include a tape, voice chip or other sound producing means in order to produce desired sounds, such as speech, breathing, moaning, shouting, etc. The sound producing device may include a wireless headphone option (FIG. 16 and accompanying text).

About the exterior of the psuedo skull 301, a layer of gelatinous elastomer 318. The gelatinous elastomer 318 may be formed into appropriate structures to present a likeness of a human, such as ears 102c, forehead 319, nose 320, lips 321, etc.

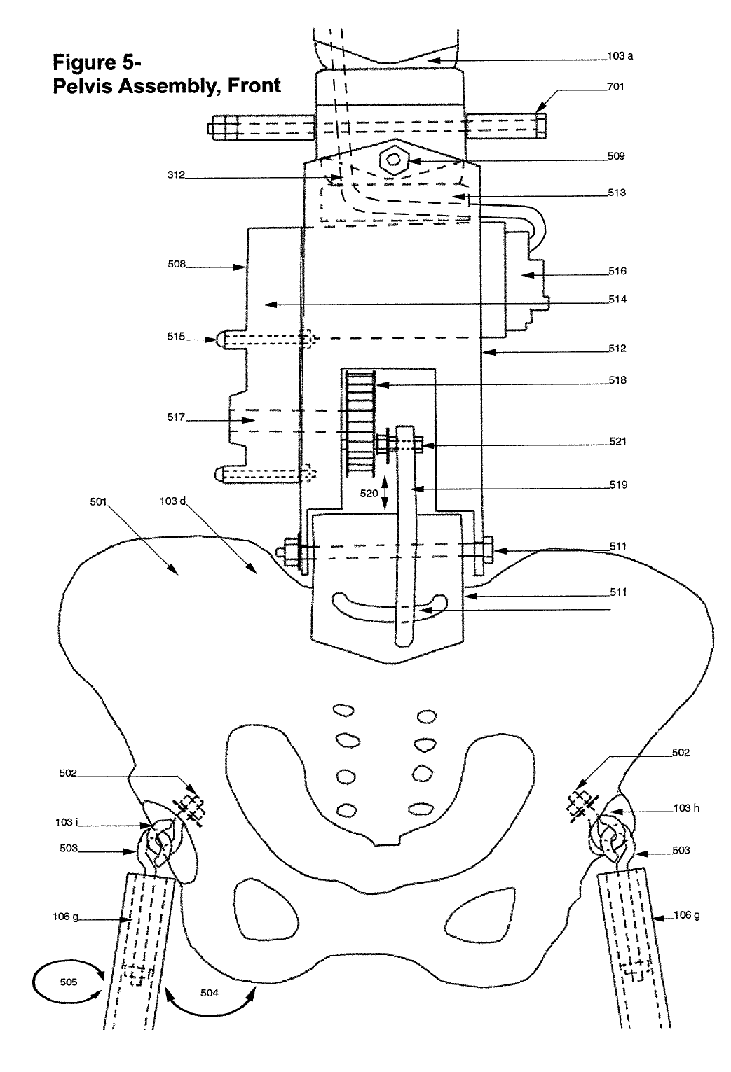

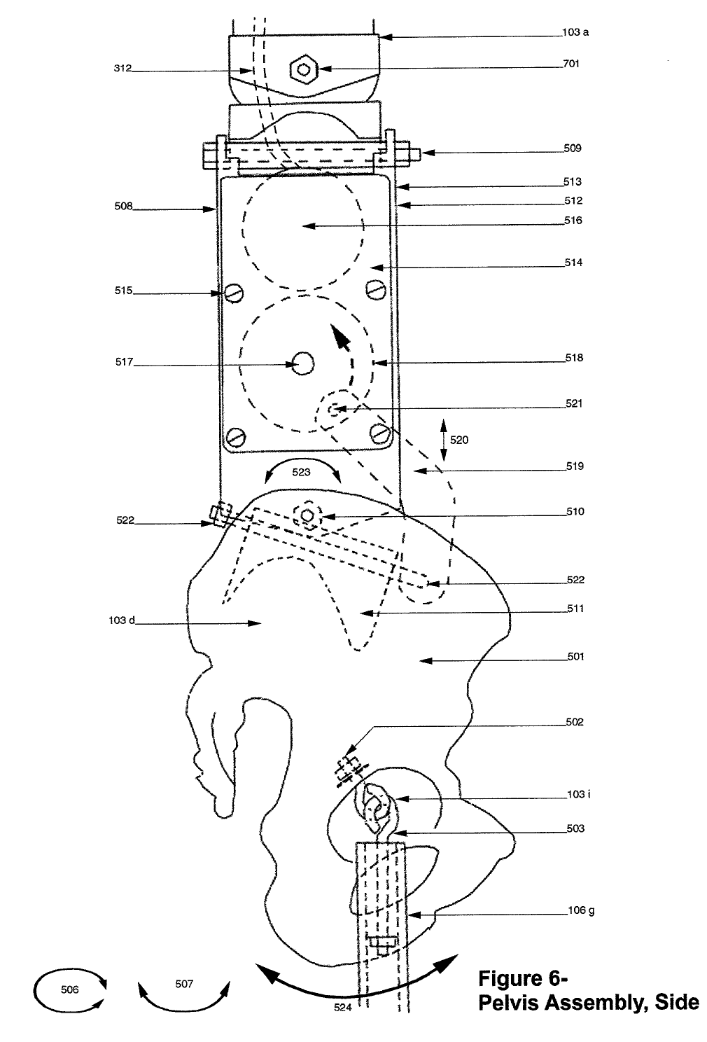

Referring to FIGS. 5 and 6, front and side views of a pelvis assembly useful in the surrogate sexual partner invention are depicted. A pelvis assembly 103d is provided. Preferably, a pseudo pelvic bone 501 will be provided having approximately the same size and shape as a human pelvic bone. The pseudo pelvic bone 501 may be made from metal, plastic, ceramic, fiber reinforced plastic, and other appropriate materials. In a preferred embodiment, CP7311 Epoxy Molding Compound, such as that available from Cosmic Plastics, Inc. of Valencia, Calif. is used because it has a tensile strength (ultimate) of almost 25,000 psi. In addition, its processing temperature is low, but it can withstand the heat of having molten gel cast around it.

The pelvic assembly 103d includes hip joints 103h and 103i. The hip joints include a pair of attached eye bolts 502 and 503. The eye bolts 502 and 503 are mutually attached to the eye of the other, and provide motion of a pseudo leg bone 106b proximally and distally with respect to the pseudo pelvic bone 501 as indicated by arrow 504 and rotationally as indicated by arrow 505 in FIG. 4, and as indicated by arrows 506 and 507 respectively on FIG. 6, in order to approximate leg motion with respect to a natural hip joint.

Attached to the pelvis assembly 103d is a pelvis actuator assembly 508. The pelvis actuator has an attachment means 509 such as a nut and bolt at its top for attachment to the spine 103a. At the bottom of the pelvis actuator assembly 508, there is an attachment means 510 for attaching to a mount 511 on the pseudo pelvic bone 501. The pelvis actuator assembly 508 includes a motor housing 512 that has an outer case 513. The outer case 513 may be an appropriate rigid structure such as 3 inch by 3 inch by ⅛ inch square aluminum tubing. A motor 514 may be included in or on the outer case 513. The motor may be any appropriate motor for the application of providing reciprocating pelvic motion. One such appropriate motor is a permanent magnet DC CYMS-G2602-730 available from Merkle-Korff Industries of Des Plaines, Ill. The motor 514 may be attached to the outer case 513 by an appropriate structure such as bolts 515. The motor is electrically connected to the cord 312 and transformer (not shown) in order to receive safe DC power and provide the power to motor windings 516. When powered, the motor 514 causes a motor shaft 517 to turn, which rotates a pulley or gear 518. The pulley or gear 518 may be any appropriate pulley or gear, such as a 2.5 inch diameter bronze pulley. There is an angled lever 519 attached to the pulley at a location other than the pulley center. The angled lever 519 as depicted includes about a 135 degree angle in it, but depending on the implementation, a straight lever, an L-shaped lever, or a lever of another configuration may be used. By attaching one end of the lever 519 to the pulley 518 at a location other than the pulley center, as the pulley rotates, the lever is lever is pulled up and down as indicated by arrow 520. At the attachment point of the lever 519 to the pulley 518 there is an appropriate lever pivot 521 such as a nut and bolt, a pin, a rivet, etc.

At the opposite end of the lever 519 from the lever pivot 521, the lever 519 is attached to a fastener 522 which affixes the lever 519 to the mount 511 and thus to the pseudo pelvic bone 501. By virtue of that attachment 522, when the lever moves up and down as indicated by arrow 520, the top of the pseudo pelvic bone 501 will be rocked back and forth as indicated by arrow 523. This in turn will cause a reciprocal rocking or moving of the bottom of the pelvic bone 501 as indicated by arrow 524. Such reciprocating motion of the pelvis causes movement of the vaginal cavity 103e in order to provide pleasure to a user when a male organ is inserted therein.

An advantage of using a realistic pelvic bone (modeled after an actual female pelvic bone, reinforced as necessary) is that it allows the entire pelvic region to move realistically when actuated by the motor. It also keeps the gel in this region (which has a large cross-sectional area) free from distortion. It provides hip bones below the skin surface, as well as a pubic bone. Naturally, there is sufficient room to accommodate a male organ both vaginally and anally in any position and angle capable in intercourse.

The reader should also note that FIGS. 5 and 6 depict a rib fastener assembly 701 as described in greater detail herein.

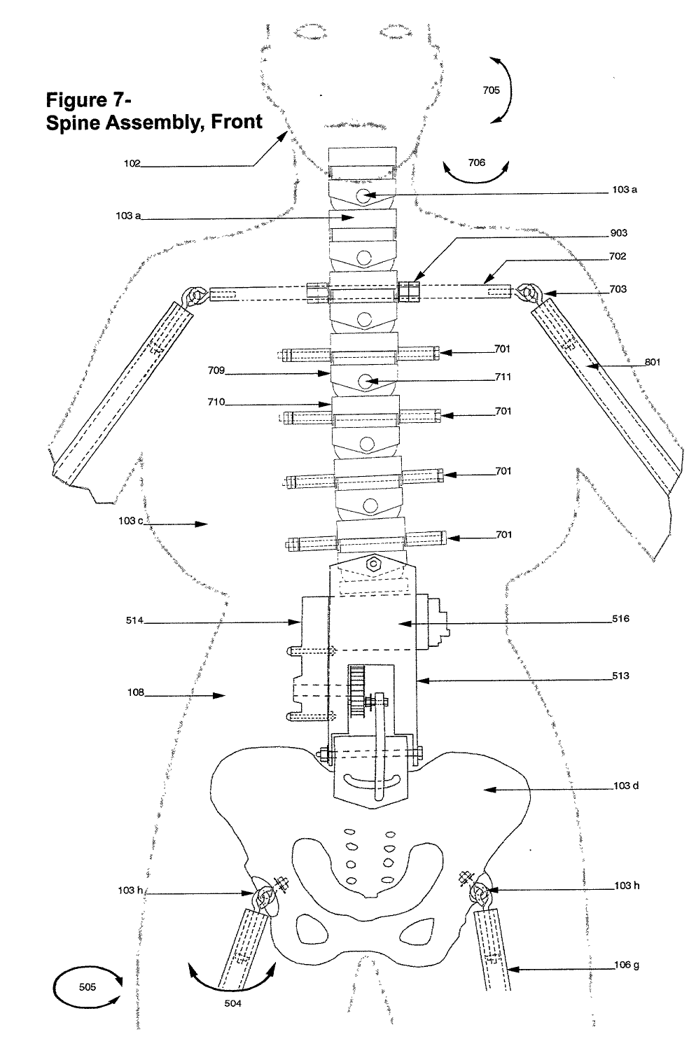

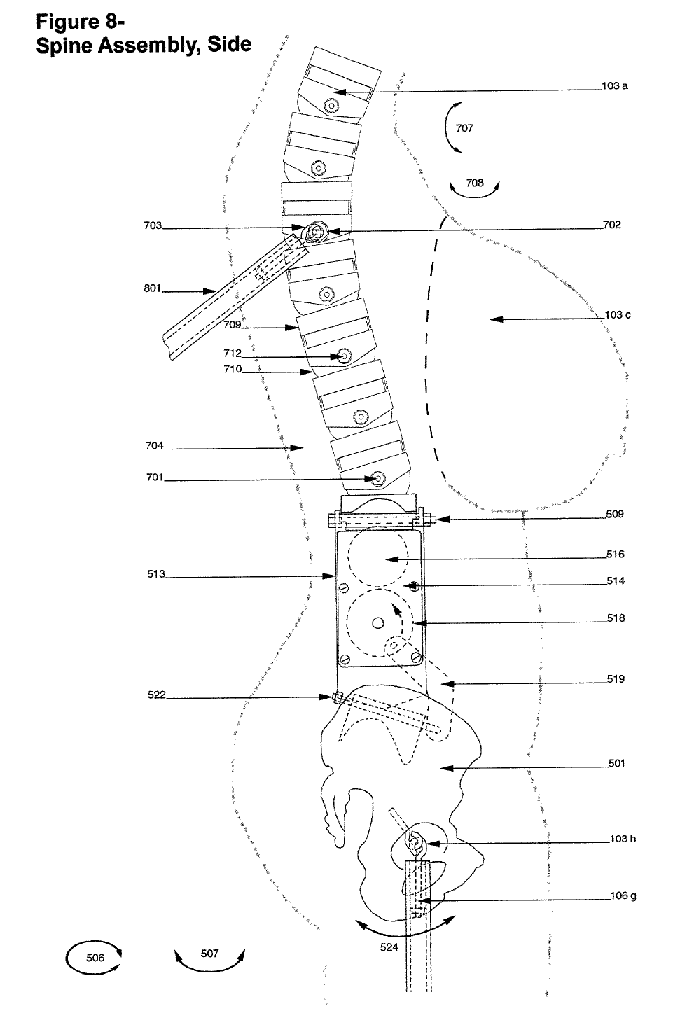

Referring to FIGS. 7 and 8, front and side views of the surrogate sexual partner depicting the spine assembly 103a are provided. The spine assembly 103a extends generally between the head assembly 102 and the pelvis assembly 103d. At appropriate points on the spine assembly 103a, there are rib fastener assemblies 701 for attaching ribs (not shown) thereto. The rib fastener assemblies 701 may include any parts appropriate for fastening ribs to the spine 103a, such as nuts, bolts, washers and otherwise. Also at an appropriate point on the spine 103a, there is a shoulder fastener assembly 702. The shoulder fastener assembly 702 may include appropriate structures such as nuts, rods, etc. in order to affix a shoulder joint 703 thereto.

The spine 103a may be constructed from any structures or materials which provide desired motion in 3 dimensions in order to replicate flexibility of a human spine, as indicated by arrows 705, 706, 707 and 708. This may be achieved by use of a specialized chain, such as an IGUS® Triflex Energy Chain, Series 333-50-250 plastic energy chain (2.5 inch. triaxial version) available from Igus, Inc. of East Providence, R.I. The preferred chain has square links 709 and 710 in which the pin and links 711 and 712 are offset by 90 degrees in order to provide flexing in all 3 dimensions. In one embodiment of the current design, after the power cord is drawn through it, the Triflex Energy Chain can be temporarily imobilized in an “S”-curved shape (like a human spine) and filled with a firm Kraton in order to increase spine stiffness and shape memory. Other materials may be used for a spine, including semi-rigid formable materials, such as Nitinol wire.

Pseudo muscle tissue 704 such as may be produced by using a firm gelatinous elastomer is depicted. Fatty tissue for the breasts 103c such as may be producing using a soft gelatinous elastomer is depicted.

An arm 801 of pseudo bone such as 0.5 inch diameter natural hard fiber tube is depicted.

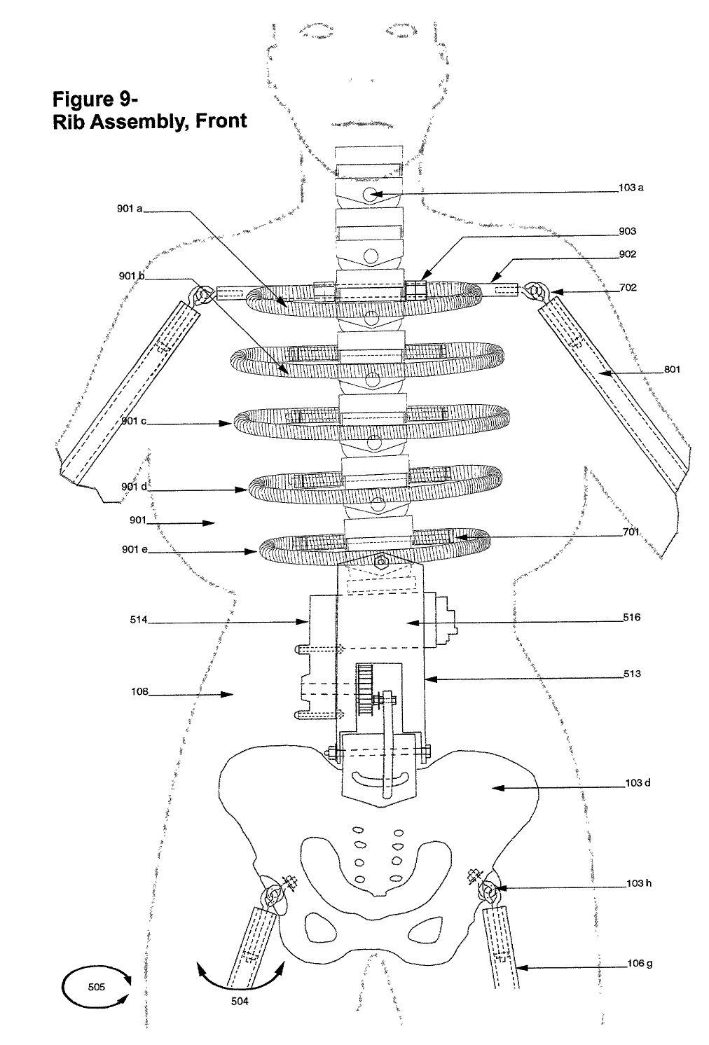

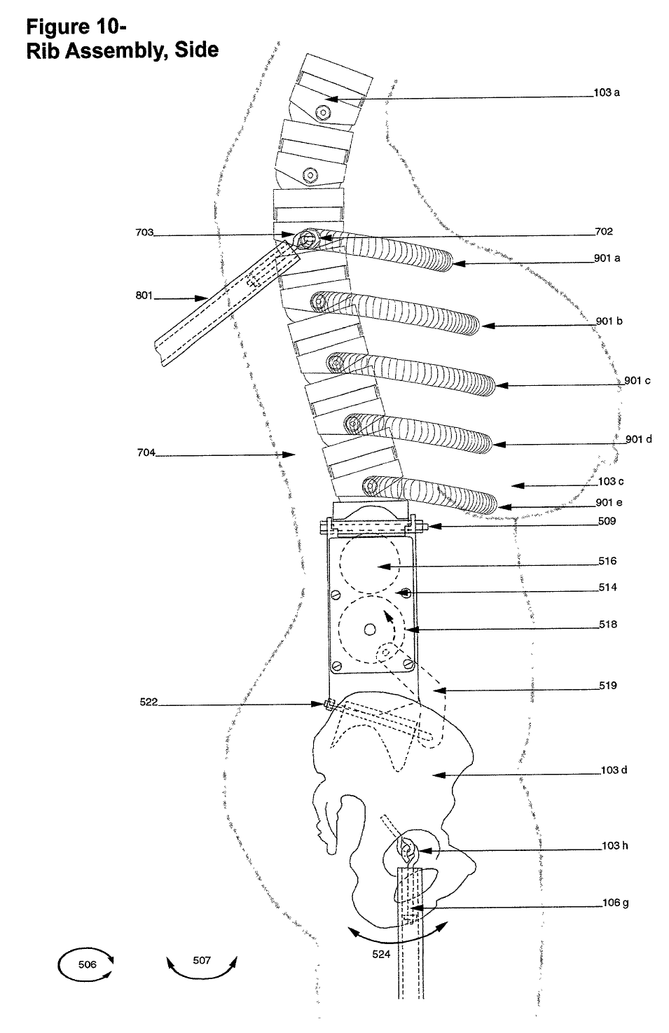

Referring to FIGS. 9 and 10, front and side views of a rib assembly useful in the surrogate sexual partner are depicted. The rib assembly 901 includes a plurality of individual ribs 901a, 901b, 901c 901d and 901e. Each rib 901a-901e is preferably constructed from a semi-rigid yet flexible and resilient material, such as steel, plastic, composite, rubber, elastomer, or otherwise. A preferred rib is made from 0.75 inch outside diameter stainless steel extension spring having 11 coils per inch in order to approximate the rigidity, resilience and flexibility of ordinary human ribs and provide a lifelike feel. The ribs 901a-901e are attached to the spine 103a by use of a rib fastener 701 which may include nuts and bolts, pins, screws, rivets, clips or other fastening devices.

FIG. 9 also depicts a shoulder mount structure 902 to which a shoulder joint 702 and arm 801 may be mounted. The shoulder mount structure 902 extends from and is fastened to the spine 103a by a fastener mechanism 903 such as nuts and bolts or other appropriate fasteners.

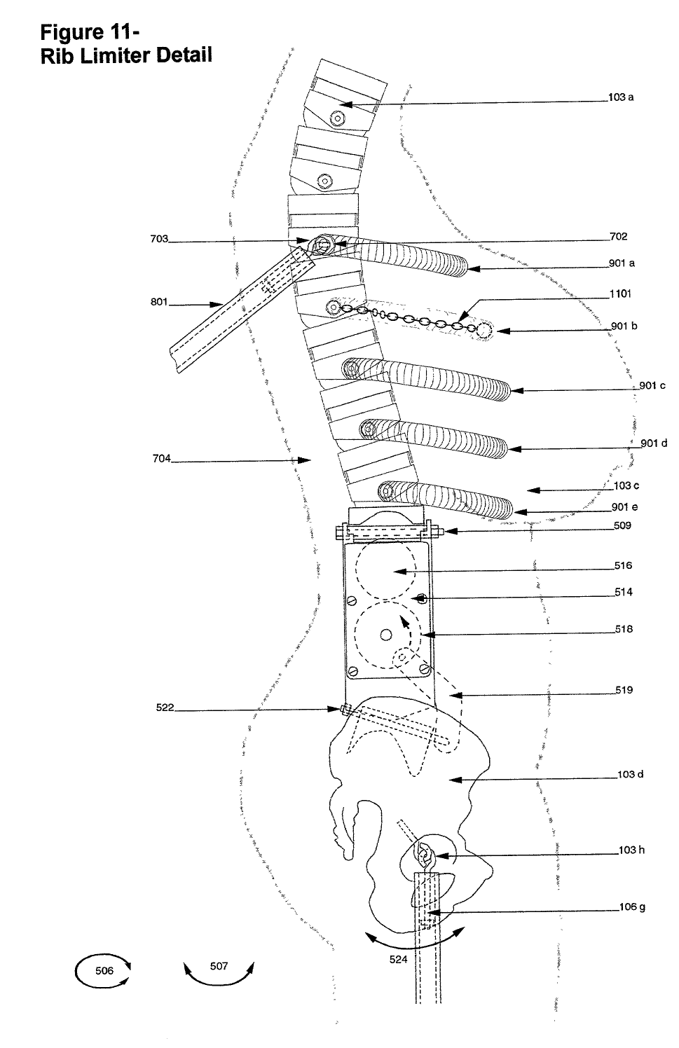

Referring to FIG. 11, it can be seen that each rib such as 901b may include a rib limiting chain 1101 extending from the spine 103a to a far point on the outer circumference of the rib 901b. Then length of the rib limiting chain 1101 may be chosen to limit the rib's greatest dimension to the front of the torso of the surrogate sexual partner, or to keep the rib under desired tension.

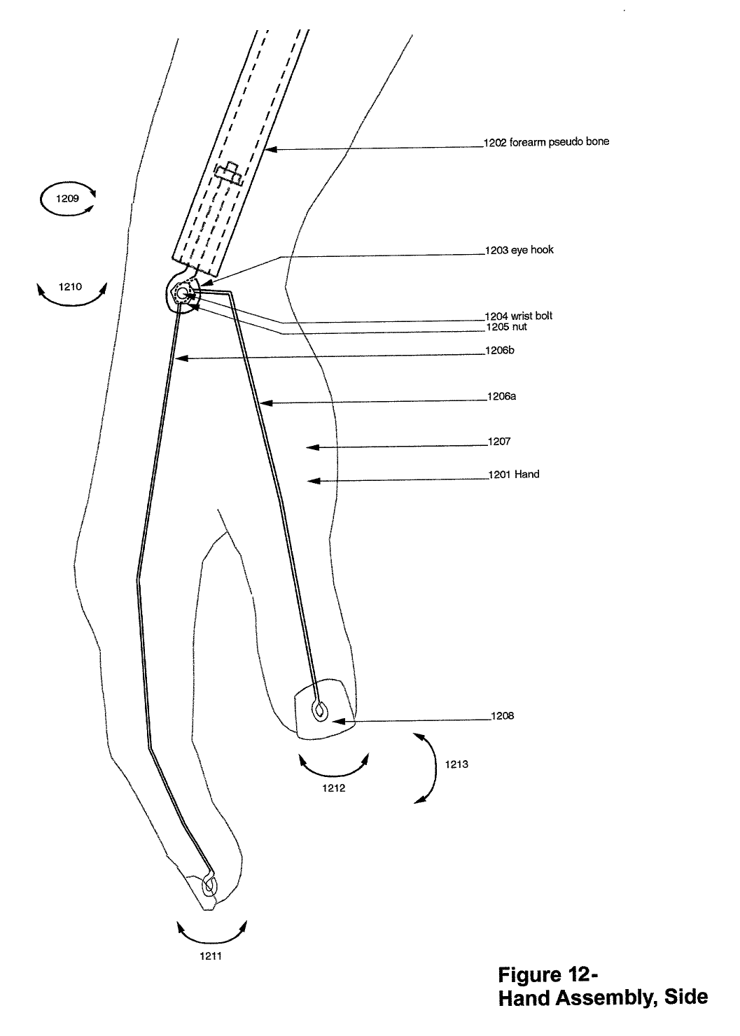

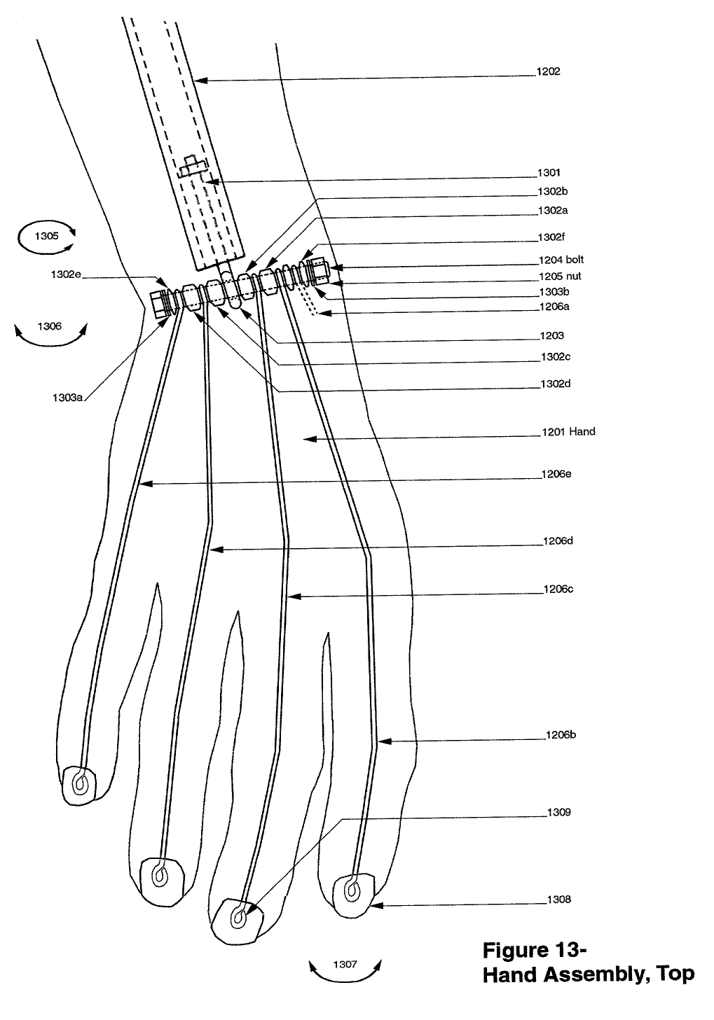

Referring to FIGS. 12 and 13, side and top views of a hand assembly of the invention is depicted. The hand assembly 1201 is attached to a forearm at forearm pseudo bone 1202 by us of an eye bolt assembly 1301 having an eye hook 1203 for use as a wrist joint. The wrist joint has a wrist bolt 1204 and wrist nut 1205 of holding the wrist assembly together extending from the wrist joint is at least one thumb 1206a structure and finger structure 1206b, surrounded by hand pseudo flesh 1207 of an appropriate gel, and preferably covered with an appropriately colored gel skin. Fingernails and thumbnails 1208 may be provided. The wrist joint allows rotational movement about the longitutional axis of the forearm 1202 as indicated by arrow 1209, and angular movement of the hand with respect to the forearm as indicated by arrow 1210. Fingers enjoy angular movement with respect to the wrist joint as indicated by arrow 1211 and the thumb enjoys two types of angular movement as indicated by arrows 1212 and 1213 with respect to the wrist joint.

Referring more particularly to FIG. 13, greater detail of the hand and wrist joint may be seen. Finger structures 1206b-1206e can be seen extending from the wrist joint to an area near the finger tip. Between each finger structure 1206b-1206e where it attached to the wrist joint, a spacer 1302a-1302e is provided. Washers 1303a and 1303b are provided at either extreme end of the wrist joint. Movement of the hand with respect to the forearm is provided at the wrist as indicated by arrows 1305 and 1306. Movement of the fingers with respect to the wrist is provided as indicated by arrow 1307. Fingernails 1308 are optionally provided. Each finger structure may be looped 1309 at its end.

Finger structures or finger bones may be fabricated from 0.08 to 0.1 inch diameter nitinol memory wire available from the Nitinol Development Corporation of Fremont, Calif. The diameter of the wire chosen depends on the rigidity of the hand grip desired when the fingers are posed. The advantage of the material nitinol is that it is extremely fatigue resistant and flexible.

The end of each finger wire is bent into a tight loop in order to anchor the acrylic fingernails which will be affixed later.

NITINOL (an acronym for Nickel Titanium Naval Ordinance Laboratory) is a family of intermetallic materials which contain a nearly equal mixture of nickel (55 wt. %) and titanium. Other elements can be added to adjust or “tune” the material properties. Nitinol exhibits unique behavior. The two terms used to describe this behavior are shape memory and superelasticity. Shape memory effect describes the process of restoring the original shape of a plastically deformed sample by heating it. This is a result of a crystalline phase change known as “thermoelastic martensitic transformation”. Below the transformation temperature, nitinol is martensitic. The soft martensitic microstructure is characterized by “self-accomodating twins”, a zigzag like arrangement. Martensite is easily deformed by de-twinning. Heating the material converts the material to its high strength, austenitic condition (see atomistic model). The transformation from austenite to martensite (cooling) and the reverse cycle from martensite to austenite (heating) does not occur at the same temperature. There is a hysteresis curve for every nitinol alloy that defines the complete transformation cycle. The shape memory effect is repeatable and can typically result in up to 8% strain recovery. Martensite in nitinol can be stress induced if stress is applied in the temperature range above Af(austenite finish temperature). Less energy is needed to stress-induce and deform martensite than to deform the austenite by conventional mechanisms. Up to 8% strain can be typically accommodated by this process. Since austenite is the stable phase at this temperature under no-load conditions, the material springs back to its original shape when the stress is removed (see atomistic model). This extraordinary elasticity is also called pseudoelasticity or transformational superelasticity. The typical stress-strain curve of a properly processed Nitinol alloy shows the loading and unloading plateaus, recoverable strain available, and the dependence of the loading plateau on the ambient temperature. The loading plateau increases with the ambient temperature. As the material warms above the austenite finish temperature, the distinctive superelastic “flag” curve is evident. Upon cooling, the material displays less elasticity and more deformation until it is cooled to where it is fully martensite; hence, exhibiting the shape memory property and recovering its deformation upon heating. Nitinol alloys are superelastic in a temperature range of approximately 50 degrees above the austenite finish temperature. Alloy composition, material processing, and ambient temperature greatly effect the superelastic properties of the material. Fortunately for the medical device community, binary nitinol alloys when processed correctly, are at their optimum superelastic behavior at body temperature.

As described above, the surrogate sexual partner has a fully articulable skeleton in order to substantially replicate the flexibility and movement of a human being.

Any of the pseudo bone structures herein may be made from any suitably rigid and flexible materials, including plastic, teflon tubing, PVC, fiber reinforced composite, hardened resin, natural hard fiber tubing, wood, metal, composites, and other materials.

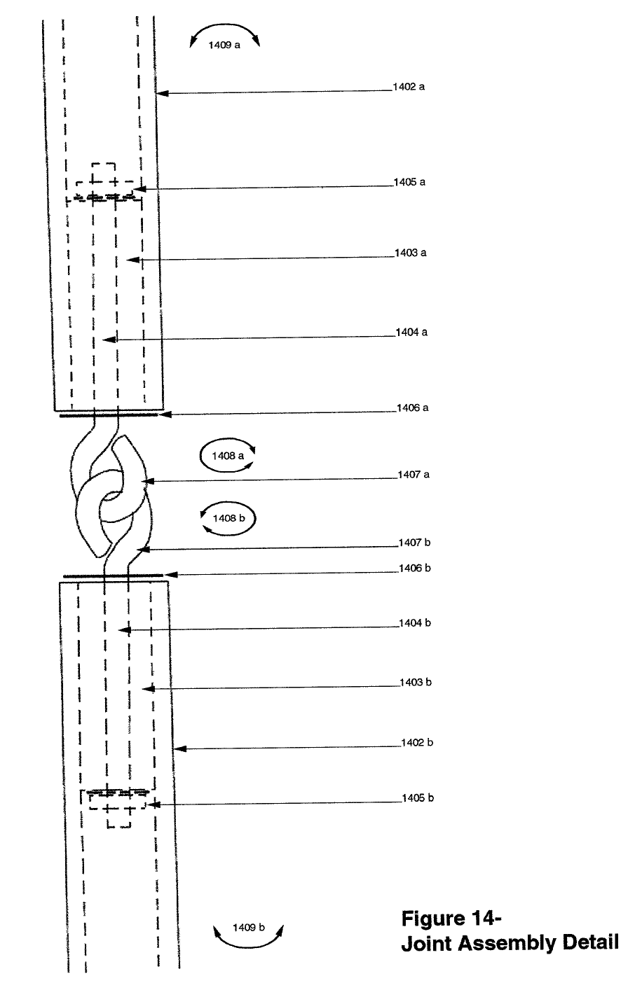

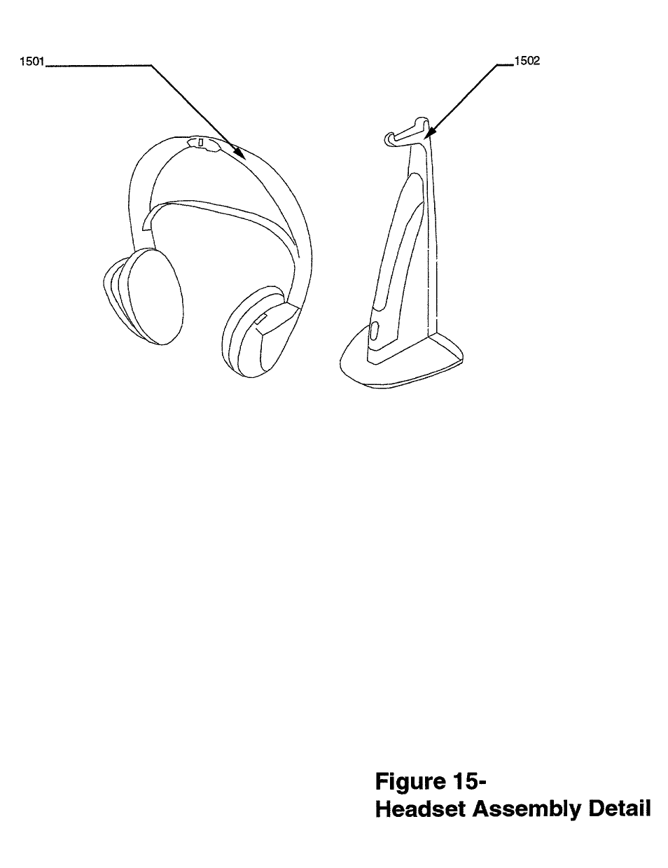

Referring to FIG. 14, a joint assembly used in some embodiments of the surrogate sexual partner is depicted. The joint assembly 1401 includes a first pseudo bone portion 1402a and a second pseudo bone portion 1402b. The first and second pseudo bone portions 1402a and 1402b are formed to receive an end plug 1403a and 1403b in their ends and to have an eye bolt 1404a and 1404b bolted therein by a nut and washer assembly 1405a and 1405b On the outer end of the pseudo bone portions 1402a and 1402b there are movement-limiting washers 1406a and 1406b. The eye bolts 1404a and 1404b each have an eye end 1407a and 1407b mutually fastened through each other in order to provide respective motion as indicated by arrows 1408a and 1408b. As a result of the movement at the eye bolts, the pseudo bone portions 102a and 1402b can move as indicated by arrows 1408a and 1409b. This structure enables the surrogate sexual partner to replicate natural human movement in the areas of the wrist, elbow, shoulder, hip, knee and ankle joints.

The knee, elbow, wrist, shoulder, knee and hip joints are formed by the joining of two eye bolts. The ankle to calf, and hand to wrist connections formed by single eye bolt each. Articulation of eye bolts in these locations approximates natural range of motion of a human being and provides for natural positioning of the surrogate sexual partner and natural response of the surrogate sexual partner to pressure or movement. The gel surrounding the joints acts with elastic properties to return the joint to its approximate original resting position once the deforming force has ceased.

As desired, the gelatinous elastomer of the surrogate sexual partner may be scented with any desired scenting material, whether to increase arousal, provide realism such as the use of perspiration scents, or to provide artificial scents such as perfume/cologne, in order to provide a user with olfactory stimulation.

Also as desired, the gelastinous elastomer of the surrogate sexual partner may be colored, pigmented or even painted in order to achieve a skin tone appropriate for the race and complexion desired in the surrogate sexual partner.

While the present invention has been described and illustrated in conjunction with a number of specific embodiments, those skilled in the art will appreciate that variations and modifications may be made without departing from the principles of the invention as herein illustrated, as described and claimed. The present invention may be embodied in other specific forms without departing from its spirit or essential characteristics. The described embodiments are considered in all respects to be illustrative and not restrictive. The scope of the invention is, therefore, indicated by the appended claims, rather than by the foregoing description. All changes which come within the meaning and range of equivalence of the claims are to be embraced within their scope.

1. A surrogate sexual partner comprising:
an elongate spine structure having a first end and a second end, said elongate spine structure permitting three dimensional movement within a desired range of motion,
a head assembly at said first end of said elongate spine structure,
a pseudo skull located in said head assembly,
eye sockets located in said pseudo skull,
a receptacle in said pseudo skull for receiving a sound producing device,
a sound producing device located in said receptacle,
a jaw bone attached to said pseudo skull at a pivot point, said jaw bone being pivotable with respect to said pseudo skull at said pivot point,
an oral cavity formed in said head assembly,
pseudo flesh formed on said pseudo skull to provide the appearance of a forehead, a nose, lips, ears, and skin,
a torso through which said spine structure projects,
a plurality of flexible ribs attached to said pseudo spine in the vicinity of said torso,
a shoulder mount attached to said spine structure,
a shoulder joint located on said shoulder mount,
an arm attached to said shoulder joint,
a pelvis reciprocator assembly attached to said spine second end,
an electric motor in said pelvis reciprocator,
an electrical cord attached to said electric motor for transporting electrical power to said electric motor,
a motor shaft on said electric motor,
a pulley attached to said electric motor,
a lever attached to said pulley at a location other than said pulley center,
a pseudo pelvic bone,
a mount on said pseudo pelvic bone, said mount being attached to said lever,
a vaginal cavity in the vicinity of said pseudo pelvic bone,
an anal cavity in the vicinity of said pseudo pelvic bone,
a hip joint located on said pseudo pelvic bone,
a leg attached to said hip joint,
pseudo muscle made from firm gelatinous elastomer located on said arms and legs,
pseudo mucous membranes made from a soft gelatinous elastomer located in said oral cavity, said vaginal cavity and said anal cavity, and
wherein rotation of said pulley by said motor causes said lever to engage in a reciprocating motion which in turn causes said pseudo pelvic bone to engage in a reciprocating motion.

2. A device as recited in claim 1 further comprising a vent hole in said oral cavity.

3. A device as recited in claim 1 further comprising a rib limiter chain attached to at least one of said ribs, said rib limiter chain serving to restrict movement of said rib.

4. A device as recited in claim 1 wherein at least one of said ribs comprises a coil spring.

5. A device as recited in claim 1 wherein said hip joint and said shoulder joint each comprise two eye bolts mutually fastened through each other.

6. A device as recited in claim 1 further comprising pseudo flesh covering the entirety of said surrogate sexual partner.

7. A device as recited in claim 6 wherein at least some of said pseudo flesh comprises a gelatinous elastomer that includes an A-B-A triblock copolymer compounded with a plasticizer.

8. A device as recited in claim 1 wherein at least some of said pseudo flesh comprises a gelatinous elastomer that includes about 100 parts by weight of a high viscosity triblock copolymer; and from about 200 to about 1,600 parts by weight of a plasticizing oil.

9. A device as recited in claim 1 wherein at least some of said pseudo flesh comprises a gelatinous elastomer that includes (a) 100 parts by weight of one or more high viscosity block copolymer; (b) from about 300 to about 1,600 parts by weight of a plasticizing oil; (c) a bleed reducing agent that tends to decrease bleed of said plasticizing oil from said block copolymer.

10. A device as recited in claim 1 wherein at least some of said pseudo flesh comprises a gelatinous elastomer that includes a copolymer and a plasticizing agent, said copolymer being selected from the group consisting of hydrogenated poly(isoprene), hydrogenated poly(butadiene), hydrogenated poly(isoprene+butadiene), poly(ethylene/propylene) or hydrogenated poly(ethylene/butylene+ethylene/propylene), poly(styrene-butadiene-styrene), poly(styrene-butadiene), poly(styrene-isoprene-styrene), poly(styrene-isoprene), poly(styrene-ethylene-propylene), poly(styrene-ethylene-propylene-styrene), poly(styrene-ethylene-butylene-styrene), poly(styrene-ethylene-butylene), poly(styrene-ethylene-propylene)n, poly(styrene-ethylene-butylene)n, maleated poly(styrene-ethylene-propylene-styrene), maleated poly(styrene-ethylene-butylene-styrene), maleated poly(styrene-ethylene-butylene), maleated poly(styrene-ethylene-propylene)n, maleated poly(styrene-ethylene-butylene)n, polystyrene, polybutylene, poly(ethylene-propylene), poly(ethylene-butylene), polypropylene, polyethylene, polyethyleneoxide, poly(dimethylphenylene oxide), copolymers of trifluoromethyl-4,5-difuoro-1,3-dioxole and tetrafluoroethylene, tetrafluoroethylene, polycarbonate, ethylene vinyl alcohol copolymer, polyamide and polydimethylsiloxane; wherein n is an integer.

11. A surrogate sexual partner comprising:
an articulable skeleton, said articulable skeleton including
a spine movable in 3 dimensions in order to approximate the movement and flexibility of a human spine,
a skull attached to said spine,
shoulder joints mounted to said spine,
arms attached to said shoulder joints, said arms including pseudo bone,
elbow joints on said arms,
hands attached to said arms at wrist joints,
a pelvis,
a cavity in the vicinity of said pelvis being shaped and sized to accommodate the insertion of a male sex organ therein,
hip joints attached to said pelvis,
legs attached to said hip joints, said legs including psuedo bone,
feet attached to said legs at ankle joints, and
ribs attached to said spine,
pseudo flesh covering said skeleton, at least some of said pseudo flesh including an A-B-A triblock copolymer plasticized with a plasticizer;
wherein at least one of said joints approximates the movement and flexibility of its human counterpart;
wherein at least one of said joints includes mutually fastened double eye bolts.

12. A device as recited in claim 11 wherein at least some of said pseudo bone includes a material selected from the group consisting of plastic, metal, wood and ceramic.

13. A device as recited in claim 11 wherein said plasticizer is an oil.

14. A device as recited in claim 11 wherein said A-B-A triblock copolymer is selected from the group consisting of hydrogenated poly(isoprene), hydrogenated poly(butadiene), hydrogenated poly(isoprene+butadiene), poly(ethylene/propylene) or hydrogenated poly(ethylene/butylene+ethylene/propylene), poly(styrene-butadiene-styrene), poly(styrene-butadiene), poly(styrene-isoprene-styrene), poly(styrene-isoprene), poly(styrene-ethylene-propylene), poly(styrene-ethylene-propylene-styrene), poly(styrene-ethylene-butylene-styrene), poly(styrene-ethylene-butylene), poly(styrene-ethylene-propylene)n, poly(styrene-ethylene-butylene)n, maleated poly(styrene-ethylene-propylene-styrene), maleated poly(styrene-ethylene-butylene-styrene), maleated poly(styrene-ethylene-butylene), maleated poly(styrene-ethylene-propylene)n, maleated poly(styrene-ethylene-butylene)n, polystyrene, polybutylene, poly(ethylene-propylene), poly(ethylene-butylene), polypropylene, polyethylene, polyethyleneoxide, poly(dimethylphenylene oxide), copolymers of trifluoromethyl-4,5-difuoro-1,3-dioxole and tetrafluoroethylene, tetrafluoroethylene, polycarbonate, ethylene vinyl alcohol copolymer, polyamide and polydimethylsiloxane; wherein n is an integer.

15. A surrogate sexual partner as recited in claim 11 wherein said gelatinous elastomer includes about 100 parts by weight of a high viscosity triblock copolymer; and from about 200 to about 1,600 parts by weight of a plasticizer.

16. A surrogate sexual partner as recited in claim 11 further comprising a motorized pelvis reciprocator assembly, said pelvis reciprocator assembly serving to provide a reciprocating motion to said pelvis.

17. A surrogate sexual partner comprising:
an articulable skeleton,
structures to approximate the likeness of a human being, including arms, legs, hands, feet, and a torso,
at least one body orifice, said body orifice having a lining of soft gelatinous elastomer in order to approximate the feel of natural human mucous membrane,
pseudo flesh covering said skeleton, said pseudo flesh including an A-B-A triblock copolymer plasticized with a plasticizing agent.

18. A surrogate sexual partner as recited in claim 17 wherein said A-B-A triblock copolymer is selected from the group consisting of hydrogenated poly(isoprene), hydrogenated poly(butadiene), hydrogenated poly(isoprene+butadiene), poly(ethylene/propylene) or hydrogenated poly(ethylene/butylene+ethylene/propylene), poly(styrene-butadiene-styrene), poly(styrene-butadiene), poly(styrene-isoprene-styrene), poly(styrene-isoprene), poly(styrene-ethylene-propylene), poly(styrene-ethylene-propylene-styrene), poly(styrene-ethylene-butylene-styrene), poly(styrene-ethylene-butylene), poly(styrene-ethylene-propylene)n, poly(styrene-ethylene-butylene)n, maleated poly(styrene-ethylene-propylene-styrene), maleated poly(styrene-ethylene-butylene-styrene), maleated poly(styrene-ethylene-butylene), maleated poly(styrene-ethylene-propylene)n, maleated poly(styrene-ethylene-butylene)n, polystyrene, polybutylene, poly(ethylene-propylene), poly(ethylene-butylene), polypropylene, polyethylene, polyethyleneoxide, poly(dimethylphenylene oxide), copolymers of trifluoromethyl-4,5-difuoro-1,3-dioxole and tetrafluoroethylene, tetrafluoroethylene, polycarbonate, ethylene vinyl alcohol copolymer, polyamide and polydimethylsiloxane; wherein n is an integer.

19. A surrogate sexual partner as recited in claim 17 wherein said gelatinous elastomer includes about 100 parts by weight of a high viscosity triblock copolymer; and from about 200 to about 1,600 parts by weight of a plasticizer.

20. A surrogate sexual partner as recited in claim 17 wherein said orifice is a vaginal cavity, the surrogate sexual partner further comprising a pelvis and a motorized pelvis reciprocator assembly, said pelvis reciprocator assembly serving to provide a reciprocating motion to said pelvis.

21. A surrogate sexual partner comprising:
an articulable skeleton,
pseudo flesh covering said skeleton,
wherein said pseudo flesh includes a gelatinous elastomer material having a plasticizer and a triblock copolymer of the general configuration A-B-A;
wherein A is a hygrogenated polymer;
wherein the combined weights of said ethylene/propylene and said ethylene/butylene comprise more than about 50 weight percent of said hydrogenated polymer B;
wherein said plasticizer associates with said hydrogenated polymer B;
wherein said triblock copolymer has a measurable percent elongation at break;
wherein said plasticizer tends to increase the percent elongation at break of said triblock copolymer; and
wherein said plasticizer tends to decrease the Gram Bloom rigidity of said triblock copolymer.

22. A surrogate sexual partner as recited in claim 21 wherein said partner provides a combination of tactile, visual, auditory and olfactory stimulation to a human user.

23. A surrogate sexual partner comprising:
an articulable skeleton, said articulable skeleton including
a spine movable in 3 dimensions in order to approximate the movement and flexibility of a human spine,
a skull attached to said spine,
shoulder joints mounted to said spine,
arms attached to said shoulder joints, said arms including pseudo bone,
elbow joints on said arms,
hands attached to said arms at wrist joints,
a pelvis,
a cavity in the vicinity of said pelvis being shaped and sized to accommodate the insertion of a male sex organ therein,
hip joints attached to said pelvis,
legs attached to said hip joints, said legs including psuedo bone,
feet attached to said legs at ankle joints,
ribs attached to said spine,
pseudo flesh covering said skeleton, at least some of said pseudo flesh including an A-B-A triblock copolymer plasticized with a plasticizer;
wjereom
wherein at least one of said joints approximates the movement and flexibility of its human counterpart.

24. A surrogate sexual partner as recited in claim 22 wherein at least one of said joints includes a U-joint.

25. A surrogate sexual partner as recited in claim 22 further comprising at least one finger structure that includes nitinol wire.
